(19) United States
(12) Patent Application Publication
(10) Pub. No.: US 2020/0098466 A1
(43) Pub. Date: Mar. 26, 2020

(54) MACHINE LEARNING IMPLEMENTATIONS FOR A MENU GENERATION PLATFORM

(71) Applicant: NUTRISTYLE INC., Meridian, ID (US)

(72) Inventors: Scott Murdoch, Bend, OR (US); Todd Albro, Eagle, ID (US); Caleb Skinner, Beaverton, OR (US); Shannon Madsen, Livermore, CA (US); Lee Brillhart, Seattle, WA (US)

(21) Appl. No.: 16/556,136

(22) Filed: Aug. 29, 2019

Related U.S. Application Data
(60) Provisional application No. 62/725,155, filed on Aug. 30, 2018.

(51) Int. Cl. G16H 20/60; G16H 10/20; A23L 33/00; H04W 4/021; G06F 3/0482

(52) U.S. Cl. CPC G16H 20/60; G16H 10/20; G06F 3/0482; H04W 4/021; A23L 33/30

(57) ABSTRACT

A method of operating a machine learning model on a menu generation platform involves storing user modification for a menu generation platform comprising a menu generation algorithm, a food preference algorithm, a geofenced equivalence recommendations algorithm, and a behavior modification algorithm in a historical user interaction database. The method generates modification and optimization controls for the menu generation platform through operation of the machine learning algorithm configured by entries in the historical user interaction database and entries in a platform user interaction database. The method generates relevant suggestions and modifications for the menu generation algorithm, the food preferences algorithm, geofence equivalence algorithm, and behavior modification algorithm, displayable through a user interface.

### BACKGROUND

Improving the user experience with a food centric platform is a difficult task as user preferences, tastes, and behaviors are each dependent on a plurality of multidimensional factors that hinders the accuracy of predictions related to preferences, tastes, and behavior.

Consistent, defined period meal planning allows users to stick to their dietary constraints by limiting unplanned meals that may not fit their nutritional or caloric targets for the particular meal, day, week, and/or other defined period, or which they don't like or can't afford. While meal planning is useful, many individuals find the process of choosing meals and ingredients that fit within their dietary constraints and meet other objectives to be tedious and complex. This may be due to various reasons including finding meals that meet their food preferences, finding the ingredients for those meals, and adjusting portion sizes to meet the nutrient and caloric (kilocalorie or “kcal”) targets for the meal and/or day.

Food preferences are difficult to determine due to the many nuances associated with how different combinations of taste, texture, smell, color and previous experiences appeal to different individuals. These nuances make it difficult to generate suggestions for new recipes or dishes that other individuals may enjoy.

Adhering to a diet with caloric and nutrient goals, and that is optimized with respect to other user preferences, including food brands and grocers, is especially challenging when travelling or when in an unfamiliar place. The unfamiliarity of a new location may make it difficult for individuals to find local restaurants or grocery stores that have the foods that will meet their goals, are close enough to the user, and offer local cuisine that fits the user's diet.

Influencing individuals to make healthier dietary and related lifestyle decisions is a difficult task to accomplish and quantify. Many implementations of behavior modifying techniques that have been utilized in the past to help individuals make healthier decisions tend to be too broad and/or ineffective to appeal to individuals while lacking the resources to adequately gauge the effectiveness of the implementation.

Therefore, a need exists for improving predictions of user preferences, tastes, and behaviors on a food centric platform.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system 100 in accordance with some embodiments.

FIG. 2 illustrates a method 200 in accordance with some embodiments.

FIG. 3 illustrates a recurrent neural network 300 in accordance with some embodiments.

FIG. 4 illustrates a bidirectional recurrent neural network 400 in accordance with some embodiments.

FIG. 5 illustrates a deep bidirectional recurrent neural network 500 in accordance with some embodiments.

FIG. 6 illustrates a long short-term memory 600 in accordance with some embodiments.

FIG. 7 illustrates a basic deep neural network 700 in accordance with some embodiments.

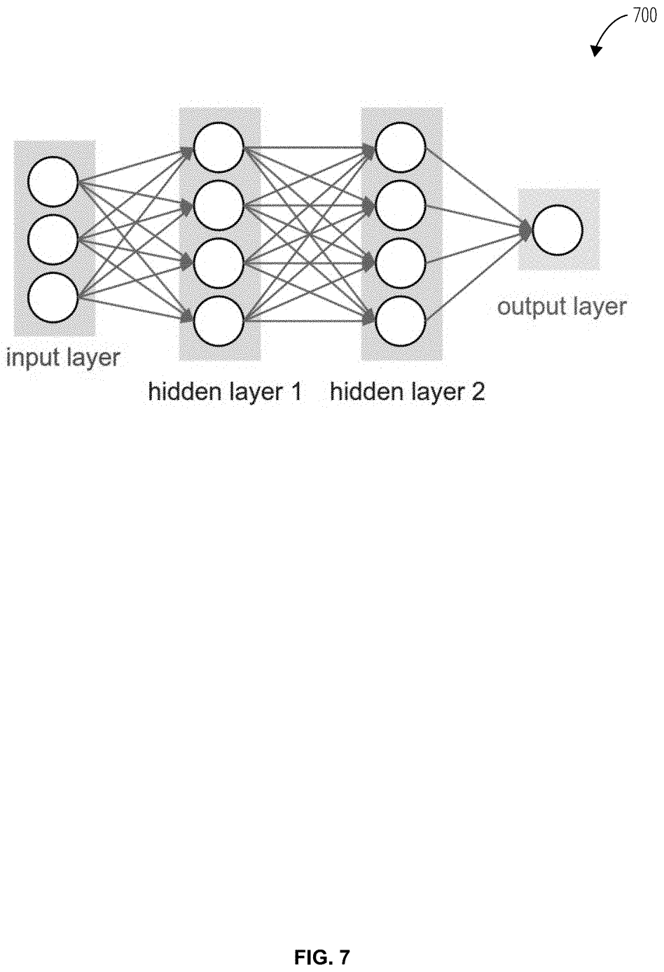

FIG. 8 illustrates an artificial neuron 800 in accordance with some embodiments.

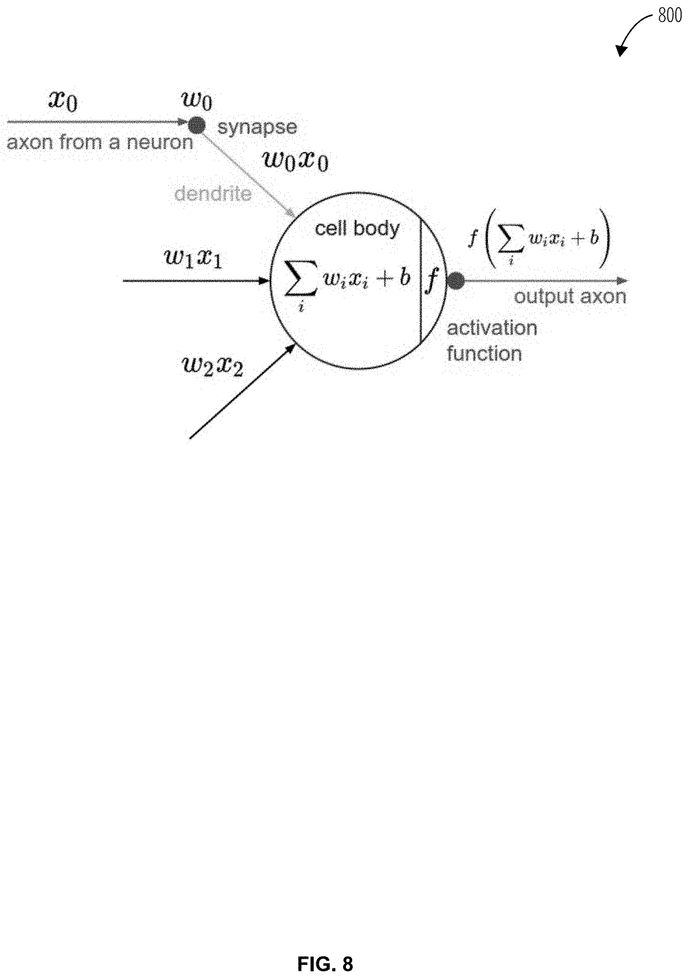

FIG. 9 illustrates a system 900 in accordance with some embodiments.

FIG. 10 illustrates a method 1000 in accordance with some embodiments.

FIG. 11 illustrates a system 1100 in accordance with some embodiments.

FIG. 12 illustrates a system 1200 in accordance with some embodiments.

FIG. 13 illustrates a system 1300 in accordance with some embodiments.

FIG. 14 illustrates a system 1400 in accordance with some embodiments.

FIG. 15 illustrates a system 1500 in accordance with some embodiments.

FIG. 16 illustrates a system 1600 in accordance with some embodiments.

FIG. 17 illustrates a system 1700 in accordance with some embodiments.

FIG. 18 illustrates a system 1800 in accordance with some embodiments.

FIG. 19 illustrates a system 1900 in accordance with some embodiments.

FIG. 20 illustrates a method 2000 in accordance with some embodiments.

FIG. 21 illustrates a system 2100 in accordance with some embodiments.

FIG. 22 illustrates a process 2200 in accordance with some embodiments.

FIG. 23 illustrates a system 2300 in accordance with some embodiments.

FIG. 24 illustrates a system 2400 in accordance with some embodiments.

FIG. 25 illustrates a system 2500 in accordance with some embodiments.

FIG. 26 illustrates a system 2600 in accordance with some embodiments.

FIG. 27 illustrates a method 2700 in accordance with some embodiments.

FIG. 28 illustrates a system 2800 in accordance with some embodiments.

FIG. 29 illustrates a system 2900 in accordance with some embodiments.

FIG. 30 illustrates a system 3000 in accordance with some embodiments.

FIG. 31 illustrates a system 3100 in accordance with some embodiments.

FIG. 32 illustrates a system 3200 in accordance with some embodiments.

FIG. 33 illustrates a system 3300 in accordance with some embodiments.

FIG. 34 illustrates a system 3400 in accordance with some embodiments.

FIG. 35 illustrates a system 3500 in accordance with some embodiments.

FIG. 36 illustrates a method 3600 for modifying behavior in accordance with some embodiments.

FIG. 37 illustrates a system 3700 in accordance with some embodiments.

FIG. 38 illustrates a system 3800 in accordance with some embodiments.

FIG. 39 illustrates a system 3900 in accordance with some embodiments.

FIG. 40 illustrates a system 4000 in accordance with some embodiments.

FIG. 41 illustrates a simplified system 4100 in which a server 4104 and a client device 4106 are communicatively coupled via a network 4102 in accordance with some embodiments.

FIG. 42 illustrates a system 4200 in accordance with some embodiments.

FIG. 43 is an example block diagram of a computing device 4300 that may incorporate embodiments of the present invention.

### DETAILED DESCRIPTION

“Gated Recurrent Unit (GRU)” refers to are a gating mechanism in recurrent neural networks. GRUs may exhibit better performance on smaller datasets than do LSTMs. They have fewer parameters than LSTM, as they lack an output gate.

“Beam Search” refers to a heuristic search algorithm that explores a graph by expanding the most promising node in a limited set. Beam search is an optimization of best-first search that reduces its memory requirements. Best-first search is a graph search which orders all partial solutions (states) according to some heuristic. But in beam search, only a predetermined number of best partial solutions are kept as candidates. It is thus a greedy algorithm. Beam search uses breadth-first search to build its search tree. At each level of the tree, it generates all successors of the states at the current level, sorting them in increasing order of heuristic cost. However, it only stores a predetermined number, β, of best states at each level (called the beam width). Only those states are expanded next. The greater the beam width, the fewer states are pruned. With an infinite beam width, no states are pruned and beam search is identical to breadth-first search. The beam width bounds the memory required to perform the search. Since a goal state could potentially be pruned, beam search sacrifices completeness (the guarantee that an algorithm will terminate with a solution, if one exists). Beam search is not optimal (that is, there is no guarantee that it will find the best solution). In general, beam search returns the first solution found. Beam search for machine translation is a different case: once reaching the configured maximum search depth (i.e. translation length), the algorithm will evaluate the solutions found during search at various depths and return the best one (the one with the highest probability). The beam width can either be fixed or variable. One approach that uses a variable beam width starts with the width at a minimum. If no solution is found, the beam is widened and the procedure is repeated.

“Loss Function” refers to as the cost function or error function (not to be confused with the Gauss error function), is a function that maps values of one or more variables onto a real number intuitively representing some “cost” associated with those values.

“ReLU” refers to a rectifier function, an activation function defined as the positive part of its input. It is also known as a ramp function and is analogous to half-wave rectification in electrical signal theory. ReLu is a popular activation function in deep neural networks.

“Sigmoid Function” refers to a function of the form f(x)=1/(exp(−x)). The sigmoid function is used as an activation function in artificial neural networks. It has the property of mapping a wide range of input values to the range 0-1, or sometimes −1 to 1.

“Hyperbolic Tangent Function” refers to a function of the form tanh(x)=sinh(x)/cosh(x). The tanh function is a popular activation function in artificial neural networks. Like the sigmoid, the tanh function is also sigmoidal (“s”-shaped), but instead outputs values that range (−1, 1). Thus, strongly negative inputs to the tanh will map to negative outputs. Additionally, only zero-valued inputs are mapped to near-zero outputs. These properties make the network less likely to get “stuck” during training.

“Softmax Function” refers to a function of the form f(xi)=exp(xi)/sum(exp(x)) where the sum is taken over a set of x. Softmax is used at different layers (often at the output layer) of artificial neural networks to predict classifications for inputs to those layers. The softmax function calculates the probabilities distribution of the event xi over ‘n’ different events. In general sense, this function calculates the probabilities of each target class over all possible target classes. The calculated probabilities are helpful for predicting that the target class is represented in the inputs. The main advantage of using Softmax is the output probabilities range. The range will 0 to 1, and the sum of all the probabilities will be equal to one. If the softmax function is used for a multi-classification model it returns the probabilities of each class and the target class will have the high probability. The formula computes the exponential (e-power) of the given input value and the sum of exponential values of all the values in the inputs. Then the ratio of the exponential of the input value and the sum of exponential values is the output of the softmax function.

“CTC loss function” refers to connectionist temporal classification, a type of neural network output and associated scoring function, for training recurrent neural networks (RNNs) such as LSTM networks to tackle sequence problems where the timing is variable. A CTC network has a continuous output (e.g. softmax), which is fitted through training to model the probability of a label. CTC does not attempt to learn boundaries and timings: Label sequences are considered equivalent if they differ only in alignment, ignoring blanks. Equivalent label sequences can occur in many ways—which makes scoring a non-trivial task. Fortunately, there is an efficient forward-backward algorithm for that. CTC scores can then be used with the back-propagation algorithm to update the neural network weights. Alternative approaches to a CTC-fitted neural network include a hidden Markov model (HMM).

“Adam optimizer” refers to an optimization algorithm that can used instead of the classical stochastic gradient descent procedure to update network weights iteratively based on training data. Stochastic gradient descent maintains a single learning rate (termed alpha) for all weight updates and the learning rate does not change during training. A learning rate is maintained for each network weight (parameter) and separately adapted as learning unfolds. Adam can be viewed as combining the advantages of two other extensions of stochastic gradient descent. Specifically, Adaptive Gradient Algorithm (AdaGrad) that maintains a per-parameter learning rate that improves performance on problems with sparse gradients (e.g. natural language and computer vision problems), and Root Mean Square Propagation (RMSProp) that also maintains per-parameter learning rates that are adapted based on the average of recent magnitudes of the gradients for the weight (e.g. how quickly it is changing). This means the algorithm does well on online and non-stationary problems (e.g. noisy). Adam realizes the benefits of both AdaGrad and RMSProp. Instead of adapting the parameter learning rates based on the average first moment (the mean) as in RMSProp, Adam also makes use of the average of the second moments of the gradients (the uncentered variance). Specifically, the algorithm calculates an exponential moving average of the gradient and the squared gradient, and the parameters beta1 and beta2 control the decay rates of these moving averages. The initial value of the moving averages and beta1 and beta2 values close to 1.0 (recommended) result in a bias of moment estimates towards zero. This bias is overcome by first calculating the biased estimates before then calculating bias-corrected estimates.

“Backpropagation” refers to an algorithm used in artificial neural networks to calculate a gradient that is needed in the calculation of the weights to be used in the network. It is commonly used to train deep neural networks, a term referring to neural networks with more than one hidden layer. For backpropagation, the loss function calculates the difference between the network output and its expected output, after a case propagates through the network.

“Food Distributor” refers to any purveyor (e.g., grocery store, grocery delivery service, etc.) that primarily offers ingredients to a user to utilize as the components of a meal, with the unit size of the ingredient being greater than the quantity required for an individual meal portion. A main difference between a food distributor and a restaurant/food service is in the quantity of the components usually exceeding the quantity required for a single meal.

“Nutrient” refers to a substance used by an organism to survive, grow, and reproduce. The requirement for dietary nutrient intake applies to animals, plants, fungi, and protists. Nutrients can be incorporated into cells for metabolic purposes or excreted by cells to create non-cellular structures, such as hair, scales, feathers, or exoskeletons. Some nutrients can be metabolically converted to smaller molecules in the process of releasing energy, such as for carbohydrates, lipids, proteins, and fermentation products (ethanol or vinegar), leading to end-products of water and carbon dioxide. Nutrients include both macronutrients and micronutrients. Macronutrients provide energy and are chemical compounds that humans consume in the largest quantities and provide bulk energy are classified as carbohydrates, proteins, and fats. Water must be also consumed in large quantities. Micronutrients support metabolism and include dietary minerals and vitamins. Dietary minerals are generally trace elements, salts, or ions such as copper and iron. Some of these minerals are essential to human metabolism. Vitamins are organic compounds essential to the body. They usually act as coenzymes or cofactors for various proteins in the body. Nutrients also include bioactive compounds and nutraceuticals, which may be compounds found in foods, are not necessarily synthesized by the body, and are not directly involved in any fundamental functions of the body, yet can alter various metabolic functions within the body to impact health or disease. Some of these nutrients may include lipoic acid, ubiquinones (e.g., CoQ10, carotenoids, phenolic compounds, and the like). Other nutrients impact the functional characteristics of foods, which is defined by how the nutrients impact the consumer. For example, foods of this type include nutrients which impact the glycemic index/load which determines the impact of the food in causing increased blood glucose and/or insulin levels and acid/alkali forming which focuses on the impact on pH levels in the blood and cells, for example.

“Food” refers to any substance consumed to provide nutritional support for an organism. For example, foods may be an assortment of consumable substances that include meats, grains, dairy products, fruits, mushrooms, vegetables, any plants, animals, insects, microbes, and any isolated or modified component of these. The foods may include condiments such as spices that may be added in combination to the aforementioned foods. Furthermore, foods may include beverages. Individual foods may be combined as components of a meal.

“Meal” refers to a single food component or combination of food components served individually or in combinations as a dish. A meal may include a dish of a variety of food components and spices accompanied by a beverage.

“Smart Health Device” refers to a user worn, carried, or otherwise connected device that collects and stores data (and may provide additional analyses) on real-time physical activity, health status, and medical/clinical bio measurements. An example of a “smart health device” is a fitness tracker

A method of operating a machine learning model on a menu generation platform involves storing user modification for a menu generation platform comprising a menu generation algorithm, a food preference algorithm, a geofenced equivalence algorithm, and a behavior modification algorithm in a historical user interaction database. The method generates modification and optimization controls for the menu generation platform through operation of the machine learning algorithm configured by entries in the historical user interaction database and entries in a platform user interaction database. The method generates relevant suggestions and modifications for the menu generation algorithm, the food preferences algorithm, geofence equivalence algorithm, and behavior modification algorithm, displayable through a user interface.

The ability to gather and perform analysis on large amounts of data that is related to foods allows the system cloud to learn about each user based on the feedback the user generates. The data can be run through a neural network and decisions for each user can be personalized to make the menu generation platform experience more intuitive and more useful for each user. For example, consider the following use cases:
Food removal—A user might not create food dislikes but might always, or with a high degree of probability, remove foods from their menu post generation. The cloud keeps track of all the menus that the user has generated as well as the edits that are made to them. This, over time, will create a rich environment of data that the cloud can be trained with. User X always removes hot peppers from their menu, User Y always removes meat from their menu during a timeframe that coincides with Lent, User Z adds cocoa to their menu around the holidays.
Food Likes—This functions much like the above. As the user enters food into the menus a dataset is created that can be mined for behavior. As the pattern develops, a neural network can start to accurately predict and inject foods into the menu post generation.
Exercise days—People like to exercise based on patterns. These patterns are enforced by trainers, fitness plans and life in general. Just like the above as users enter data neural networks can process the patterns and find the appropriate injection points.
Price sensitivity—People may not purchase various food items based on the price of the food. So, as the data is gathered the neural network can start to see what people are adding to their menus. As this dataset grows we can start to determine that people will or will not purchase a food based on its price. This makes the advertising engine much more valuable as the injected data is heavily weighted by user behavior.
Menu Presets—Menu presets allow the application to learn a lot about each user. For example, recipes can be presented to each user based on the kind of menu preset the user selects as well as the food restrictions they specify.
Feedback Loops—There are a number of other feedback loops that will be implemented as the menu generation platform database grows. Each user interaction point is an opportunity to engage machine learning and assist the user in creating a food & exercise regimen that is tailored for them based on their feedback.

In some configurations, the user meal preferences comprise food dislikes, food likes, food allergies or restrictions, meal and snack preferences (including preferred recipes), nutrient targets, weight or other personal health objectives, preferred food brands, and grocer or food distributor preferences, kcal target and meal presets. The linked user services may comprise a food tracking service and a grocery list service.

In some configurations, a user may provide presets defining which food items to utilize for some food sub categories, identify the food item as a meal component and/or part of a larger food component category. The presets may also be utilized to identify specific food items as well as particular combinations of food items that may be viewed as individual meals by themselves.

In some configurations, a user profile may include user preferences such as food preferences (e.g., likes/dislikes, which may be further broken down into preferred tastes and/or textures, smells, etc.), restrictions (e.g., allergies or disease), health objectives (e.g., lose weight), financial budget, grocer or food distributor (e.g., grocer supplier or direct-to-consumer provider in certain food distribution scenarios), preferred brands or private labels, preferred recipes, and preferred restaurants.

A method of operating a meal plan optimization algorithm may involve retrieving at least one user profile from a profile database. A menu generation algorithm may be configured with user preferences from the at least one user profile. A selector may be configured to retrieve food items from at least one proximal food database for the menu generation algorithm from the user preferences. The menu generation algorithm configured by the user preferences may be operated to set preferred food items for meal components of individual meals from the food items in the at least one proximal food database through operation of a food preference discriminator, set a non-user specified food item for a meal component of the individual meals without a preferred food item and additional food items through operation of the food preference discriminator, and update a meal total for the individual meals with the non-user specified food item. The menu generation algorithm may operate a meal target evaluator recursively, through operation of an iterator, to adjust and substitute meal components. A meal day plan may be generated from the individual meals. A kcal target evaluator may be operated to adjust the portion size for the individual meals in the meal day plan configured by the user preferences. And a food menu for at least one day displayable through a user interface associated with the at least one user profile, from meal day plans may be generated through operation of a constructor.

In some configurations, the kcal target evaluator configured by the user preferences from each user profile may be operated to adjust the portion size of the individual meals in the meal day plan for each user profile

In some configurations, the user preferences include food dislikes, food likes, food allergies or restrictions, meal preferences, nutrient targets, weight or other personal health objectives, financial budget, preferred grocers or food distributors, preferred brands or private labels, and kcal target from the at least one user profile.

In some configurations, the method of operating a meal plan optimization algorithm involves retrieving at least one user profile from a profile database, configuring the menu generation algorithm with food dislikes, food likes, meal preferences, nutrient targets, financial budget, and kcal target from the at least one user profile, configuring a selector with location information from the at least one user profile to retrieve food items from at least one proximal food database for the menu generation algorithm, operating the menu generation algorithm to set preferred food items for meal components of individual meals from the food items in the at least one proximal food database through operation of a food preference discriminator configured by the meal preferences, setting a non-user specified food item for a meal component of the individual meals without a preferred food item and additional food items through operation of the food preference discriminator configured by the food likes and the food dislikes, operating a meal target evaluator, and generating a meal day plan from the individual meals. The method of operating a meal plan optimization algorithm further involves adjusting the portion size of the food items for the individual meals in the meal day plan through operation of the kcal target evaluator configured by the kcal target as specified by the user, and generating a 1-n-day food menu, displayable through a user interface associated with the at least one user profile, from meal day plans typically grouped together as menus through operation of a constructor. The method may further involve adjusting the portion size of the individual meals in the meal day plan for each user profile through operation of the kcal target evaluator configured by the kcal target from each user profile.

In the method of operating a meal plan optimization algorithm, the meal target evaluator iteratively operates, through operation of an iterator, to calculate nutrient gaps and nutrient target ratios, gap ratio score, scores and other mathematical relationships among the food items that comprise the individual meals adjust portion size of the food items within individual meals with the non-user specified food item in response to detecting the meal total score being outside of a meal target range based on the gap ratio score, substitute the non-user specified food item with different non-user specified food item in the individual meal with the closest meal total outside the meal target range, and replace the food item having the lowest nutrient value in the individual meal with the different non-user specified food item with the closest meal total outside the meal target range.

In some instances, these collections of food items may have their portion sizes adjusted; and, to achieve acceptable meal scores, can have non-user specified food items substituted for other non-user specified food items. This process of food item substitution is managed by any of a number of mathematically driven algorithms that are used to view the food item in question, determine the footprint of the food item to best fit the requirements of the meal and, finally, to manage the process of verifying that the food item substitution is a better fit for the meal.

In some configurations, the algorithm for generating a menu for a user through a user interface involves retrieving the menu preset for the user from the user's profile. Menu presets include 1-n parameters to assist in identifying a unique menu, including, without limitation, macronutrient (e.g., fat, protein, carbohydrate, alcohol) targets, food favorites (e.g., a specific brand of yogurt), food restrictions, macronutrient settings for carbohydrate, fat, protein, and alcohol, and food dislikes. The kilocalorie (kcal) target for the user is also retrieved, as are 0-n micronutrient (e.g., amount of sodium or potassium) targets, as applicable. A check is performed to determine if additional user profiles will be involved in generating the menu. If additional profiles are included in the menu creation, the kcal target is increased to account for the additional profiles and a multiplier is determined to accommodate the additional individuals. After the adjustments have been made for the additional users, or if there are no additional users, foods are chosen. The profiles include, among other things, the user's likes and dislikes for certain foods, food restrictions, health objectives, financial budget, preferred food brands and/or private labels, and grocer or food distributor preferences. In some instances, the user may set a kcal target for individual meals, individual days of the defined period, or as the same kcal target for each day.

Once the user information has been collected, the menu generation process begins. The generated menu runs for 1-n meal cycles and may include standard meals (e.g., breakfast, lunch, dinner, and snacks (e.g., morning snack, afternoon snack, and evening snack)) that are ordered in the menu according to their chronological schedule. In some configurations, the meals may be ordered differently or based on user preference.

In some instances, previous menu configurations related to the user going over or under their predicted kcal target may be presented. When a new menu is generated, previous values are set to zero. For example, carry over of kcal, macronutrients, and micronutrients are reset to zero for a new menu. Food likes for individual meals or specific days are retrieved, as well as preset food likes, and preferred brands for individual meals or specific days. This preset allows users to specify specific foods at specific times in advance, for instance if the user exercises on certain days and needs a higher protein intake after their workout.

Components for the day/meal are retrieved at this point. The components may be a broad categorization of certain food items that go into a meal based on food preferences. Once the components are retrieved, the individual foods are retrieved for each component. The menu generation process then begins to perform an iterative process, going through each day and individual meals for that day, starting with the first day of meals until it reaches the final meal on the last day of the 1-n day defined period. The iterative or looped process involves randomly retrieving one food item per component, screening for various values included in the user profile, adding food likes to the meal, and updating the meal totals, and then determining the meal type. Depending on the meal type (e.g., traditional meals (e.g., breakfast, lunch, dinner) or snacks) the system may update values for the day or meal based on individual totals or modifications. This may occur 1-n times per meal component. If the meal type is a traditional meal (e.g., breakfast, lunch, dinner) gaps in the meals are calculated, such as the ratios of carbohydrates, fats, proteins, and alcohol. This process is then followed by a 1-n loop to adjust the portion size of the meals. If the meal type is a snack, the food item is added, and the snack totals are adjusted while adjusting the meal totals. Once the process of generating the menu is completed, the user profile is checked to determine the number (1-n) of users. If more than one user profile was included in the generation of the menu, the process creates a menu with appropriate adjustments to the portion size to reflect the kcal target of the user and then sets the previous menu's primary menu Boolean value to false. The process then creates a system food log day for each day of the menu. If more than one user profile is not involved in the menu generation, a menu is created with the appropriate adjustments to the portion sizes to reflect the kcal target for the user, and the previous menu's primary menu Boolean value is set to false removing the previous menu. The process then creates a food log day for each day of the menu.

The 1-n step loop adjustment for portion sizes of food components of a meal involves performing a gap ratio analysis from the food gaps created from the meal calculations following the addition of meal preferences and snacks. The gap ratio analysis checks the meal score. If the meal scores are correct, the system determines that the meal has been generated and then moves on to the next meal. If the meal score is not correct, and if the iteration completes without successfully hitting the meal target, the process checks the meal score. If the meal score is better than the existing value, the algorithm updates a score for the best scored meal combination with the current meal combinations score, and updates the best meal combination with the current meal combination. After completing 1-n number of iterations (e.g., 20), as optimized over time, the process determines if the best meal should be updated. If so, the process checks the meal totals again. Otherwise, the process moves on to a process for determining substitutions. The substitution process iterates through each food in the meal and does not process favorite food preferences. The process begins by removing food from the meal, followed by the first step a multistep iteration that runs in sets of 1-n until the process has iterated 1-n times. The process starts by looking for a food from the component collection that fits into the parameters within the set of iterations (loops). If a food item is found, a determination is made to see if the substitution works for the meal. If the substitution does not work, the food item is replaced with the original. If the substitution of the food item causes the meal to score better than the original, the food item is kept as a possible substitution, if the newly substituted food item makes the meal score worse, the process reverts back to the original food item. The process of reverting back to the original food item may also occur if no substitution is found.

Once the first step of the substitution is completed, and a substitution is not found, the food item is removed from the meal, the food sub category collection that the food item is part of is ordered based on the lowest value of needed macronutrients. The second step of the loop is then run where the food item is replaced by selecting a substitute food item from the ordered food sub category collection, the required calculations are then performed to determine the substitution. If the substitution does not work, a determination on the meal score is made. If the current meal score is better, the substituted food item is kept. If the current meal score is worse, the substituted food item is reverted back to the original food item and the process moves on to plan the next food item collection. If the substitution works, as determined by the meal score, the meal generation is completed, and the process moves on to the next meal.

A system running a meal plan nutrition optimization algorithm takes into account the preferences, dietary restrictions, health objectives, and financial budget set by the individual users and generates a single meal, series of meals, or 1-n day meal plan for the user. The optimization algorithm selects foods from local grocery store or food distributor inventories, considering a user's preferred food brands and private labels, and builds meals around them based on the totality of the user's preferences and the nutritional content of the selected food. The system provides users with a 1-n meal/day shopping or grocery list by taking those foods and listing the quantity of each food needed to build the list for the period defined by the user.

A user may set nutrient targets and preferences as well as set food items they want to include or exclude from their weekly menu of meals. The system then populates individual food items based on component categories to build meals that fit the user's target preferences within a narrow margin.

A user may set certain caloric and/or macronutrient and/or micronutrient targets for specific food items, meals and/or specific days and the system may then make adjustments to the individual meals to fit within the specified target within the meal or the day. The system may also obtain new targets for the day and then implement meals based on those targets. Additionally, the system may make adjustments to the meals based on a desired nutrient total/ratio that may cross reference the targets with the user's caloric target. The categorization of a meal (e.g., traditional meal (e.g., breakfast, lunch, and dinner) or a snack) may determine which targets (nutrient or kcal) are tracked and utilized in the adjustment of a daily target. For instance, the macro nutrient targets may not be utilized as a deciding factor to select a food item for a snack and may also not be utilized to influence the daily totals of the nutrient targets. However, the kcals in the snack may tracked and utilized to adjust portion sizes of the meals for that particular day.

In some configurations, as an example, the system may note that a user specified a brand or private label name soup on their profile, and then use that specified brand to find related foods items by that brand or private label to use in other meal options.

The system may also provide food variety to users by generating meals starting with a randomly selected food item. The randomly selected food item would be from a small pool of food items that meet the user's food preferences criteria, and then build a meal around that food item.

When setting up a food menu for a defined period, a user may specify a particular food item that they would like to use by entering the particular food item manually (e.g., typing or selecting a picture of the food item), by scanning the Universal Product Code (UPC) of that food item in their possession, by voice, or through other means.

By scanning the UPC, for example, the system may connect to a food item database associated with food items available at local grocery stores, local grocery store chains, or other nearby food distributors within the area, determined by the user's profile, or through location services, and determine whether the food item is in stock when generating the menu for the predetermined time period. This may be especially useful in determining whether to include seasonal food items in the user's weekly food menu. Regardless how the link to the grocer's or food distributor's inventory data is activated, food items will be identified not just by type, but by specific brand name (including private label or “store brands”) to capture the most accurate nutrition data for each food item. In addition, in certain embodiments, a local grocer or food distributor will be able to flag or otherwise identify products of which they have, for example, excess inventory, and have those items made extra visible to users through message or other process, including as a special promotion or coupon, or as a “special price” item when comparing meal and shopping list cost to the user specified financial budget. Such food promotions could be based, for example, on a specific branded or private label food item, a product category (e.g., yogurt), or a department (e.g., cheese) and the nature of the promotion(s) can be varied dynamically to assist grocers or food distributors in managing their inventory.

The system may also connect with the user's devices (e.g., wearable devices, mobile device, etc.) to determine the user's activity level. With the user's activity level, the system may be able to adjust the caloric target and/or portion size of the user's meals to more accurately reflect their dietary needs for that day.

The system may decide what foods to add to the user's menu based on an unassociated shopping list a user has created for a particular grocer. The system may view items within the list and plan meals based on the items on the list. The system may also provide users with suggestions on how to obtain the food items based on price or convenience. For example, the system may provide users with suggestions about the local grocers that may offer the food item at a lower price than other nearby grocers and/or provide the user with grocery delivery services that can deliver the food items to them.

The system may allow the user to go through their profile and set the preferences for the generated menu. A user may identify/update foods that they like for an already created menu. The system may then take those updated values and reconstruct the menu based on the user's updated preferences. The system may provide users with the ability to recalculate/reconfigure a specific meal vs the whole day by defining specific meals they would like to have ahead of time and/or by listing foods and/or food combinations that that they would like or not like to have for a particular meal. For instance, a user may specify that they would not like to have tuna for breakfast and the system may exclude tuna as a protein option for breakfast.

The food menu may also work in combination with a user food log or food tracker allowing a user to provide feedback on whether they have been eating the foods suggested by the food menu and also provide the food menu with the ability to adjust the meal options or portion size for the day in order to meet the user's nutrient targets. Users could enter data in the food log in multiple ways, including through standard keyboard entry, scanning the product code (e.g., UPC or QR), taking a picture of the food item/meal (with subsequent AI processing identifying the meal), voice, or other means.

The system may also provide users with health risk warnings letting the user know that they have consumed a specific food item or quantity that is not recommended for them. For instance, a user may set a beer as part of a snack or meal component, and the system may advise the user that beer may not be a good option for meeting their dietary targets.

In some configurations, the system selects foods based on their availability near the location of the user, then builds individual meals that meet the user's dietary constraints and food preferences. The system may also build food menus based on the number of individuals that a user is preparing food for. The system may define individual meals based on the number of users, especially if the users provide food preferences and dietary constraints. The system may go through the food preferences and dietary constraints of each user and generate a menu that would accommodate all their constraints. Additionally, the system may integrate with shopping list applications to determine food preferences and eating habits. For instance, the system may determine particular food likes and dislikes based on the foods or combination of foods that user has had on their shopping list, as well as the frequency that a user shops for those particular items.

The system may also integrate with a user's day planner or calendar and determine specific meals to offer to the user based on when the user is available. For instance, the system may determine that the user arrives at their home fairly late every day and may suggest certain meals that are easy and quick to prepare.

The system may also compare a user to other similar users based on, for example, their food preferences, dietary selections, shopping lists, consumption food logs, exercise type and schedule, food and/or brand favorites, food restrictions, etc. to suggest particular foods or avoid particular foods even though the user has not specified that they have a preference or lack thereof for that particular food item.

The system may also integrate with a food preference algorithm that identifies food pairings to suggest particular food combinations that a user may like to see in a meal.

In some configurations, the system may allow users to enter their prescription medication and supplement information and dosage schedule to help the system plan certain meals based on certain food drug interactions. For instance, grapefruit juice may inhibit the activity of certain digestive enzymes used for metabolizing orally administered medications. The system may identify the possible contraindications for the drugs listed by a user and exclude grapefruit and grapefruit juice containing foods from the user's meal plan. Additionally, the system may take into account the time when a user takes their medication to limit certain foods and/or quantities of macronutrients that may influence the efficacy of the mediation if taken within certain time period with each other. For instance, a user may take insulin and the system may identify meals to offer the user, at specific times, that may reduce the likelihood of their blood glucose level rising.

In some configurations, each daily menu may be broken down into a combination of meals with/without snack(s). A meal may be identified as group of foods that usually contains a beverage, primary food (aka a main entree), and a number of secondary foods that enhance the primary food. This collection of foods might have something sweet that would translate into a dessert. The food component make up of a meal varies as it may be different per ethnic group, region, place in the world, diet plan, etc. A snack may be identified and selected from a group of foods, typically one or two at most, that may be consumed between a meal.

In some configurations, the generation process treats the snack and meal process differently. The generation process treats the snack similarly to the meal process, with one exception: there may be usually only 1 Food Sub Category that the algorithm selects a single food item from (unlike a meal that contain several Food Sub Categories). So, the only thing that the generator process can do may be adjust the portion size of the snack. So, for a snack the process is: a while loop takes the collection of foods that comprise the snack and adjusts the portion size of each food. Each iteration of the loop retrieves a snack score, increases or decreases the portion size based on reviewing the values of the macronutrient values of the food. Once the adjustment may be made the snack may be scored. If the adjustment has caused the meal score to decrease then the adjustment may be kept and the process continues. The iterations continue until the snack score equals zero or the snack score increases as opposed to decreasing. These upper and lower targets can be adjusted based on the tuning of the generator.

In some configurations, the meal generation process may be a 1-n loop. The process may start with a collection of food components that comprise the meal. These food components may all have their portion sizes set to a typical portion amount. The first step of the process may be to determine a number of different mathematical ways of viewing the food components in relation to each other as well as in relation to the caloric target that may be being pursued. The following values may be calculated:
Carbohydrate gap=Abs(meal.CarbohydrateTarget)−meal.CarbohydrateActuals)
Fat gap=Abs(meal.FatTarget)−meal.FatActuals)
Protein gap=Abs(meal.ProteinTarget)−meal.ProteinActuals)
Ratio of the protein value to the value the meal target
Ratio of the fat value to the value of the meal target
Ration of the carbohydrate value to the value of the meal target
Carbohydrate gap percent
Fat gap percent
Protein gap percent
The ratio of carbohydrates to fat and protein
The ratio of fat to carbohydrates and protein
The ratio of protein to carbohydrates and fat
The carbohydrate variance=Abs(carbohydrateRatio−mealCarbohydarateRatio)
The fat variance=Abs(fatRatio−mealFatRatio)
The protein variance=Abs(proteinRatio−proteinRatio)

In some configurations, a ratio may be defined as the sum of the carb, fat, and protein values divided by the specific nutrient (carbohydrate, fat, protein).

Once these values have been gathered the next step in the process may be to look at the collection of food components to find the appropriate food component to adjust relative to the target that may be trying to be achieved. This process iterates through the food component collection until either a successful meal has been generated or the 1-n iteration count may be breached.

If the adjustment of the portion size does not result in the successful generation of a meal the next step may be the process may be food substitution. This food substitution logic looks at the needed ratios and then sorts the available foods by them. The process increases the size of the circle around the food ratios until a match may be found. At this point a meal score may be taken, the food may be substituted in, the portion size adjusted and the meal may be again scored. If the substitution process results in a lower score the substitution may be considered a success and may be added as part of the meal. If the meal may be now inside a value range it may be considered generated successfully. If not the original food may be swapped back in and the process starts on the next food in the food component collection.

If a food substitution may be not found with this primary substitution process and secondary one in engaged. This process seeks to find a food for substitution based off of looking for the greatest value of carbohydrate, fat, protein and then sorts of the collection of available food substitutes in the appropriate order. This process returns a food. The process of scoring the meal, adjusting the portion sizes for the foods in the food component collection, and then scoring the resulting meal. If the score of the meal may be smaller the substitution may be considered a success. If the meal score falls into a range the meal generation may be considered a success.

In some configurations, the meal generation processes may carry over differences in the carbohydrate, fat, protein targets into the subsequent meal. This process smooths the meals throughout the day and assists with trying to hit specific targets with crude adjustment buckets. This carry over process does not calculate between days. It only moves between meals of the same day.

One of skill in the art will realize that the methods and apparatuses of this disclosure describe prescribed functionality associated with a specific, structured graphical interface. Specifically, the methods and apparatuses, inter alia, are directed to a system and method for operating a meal plan optimization algorithm that reduces the number of human interactions required to generate a typical weekly menu by automatically creating meals for the user that meet their nutritional and caloric targets. One of skill in the art will realize that these methods are significantly more than abstract data collection and manipulation.

Further, the methods provide a technological solution to a technological problem, and do not merely state the outcome or results of the solution. As an example, the system for operating a meal plan optimization algorithm reduces the number of human interactions required to generate a typical weekly menu by automatically creating meals for the user that meet their nutritional and caloric targets. This is a particular technological solution producing a technological and tangible result. The methods are directed to a specific technique that improves the relevant technology and are not merely a result or effect.

Additionally, the methods produce the useful, concrete, and tangible result of the generation and display of a weekly food menu, thereby identifying each change as associated with its antecedent rule set.

Further, the methods are directed to a specifically-structured graphical user interface, where the structure is coupled to specific functionality. More specifically, the methods disclose a specific set of information to the user, rather than using conventional user interface methods to display a generic index on a computer.

A method of operating a food preference algorithm involves retrieving a meal or snack framework comprising at least one food component category from a meal framework database through operation of a meal selector configured by a preferences profile in a user profile, generating a meal profile comprising at least one food item retrieved from a food item database through operation of a food component selector configured by the meal framework and the preferences profile, operating a machine learning food preferences algorithm, and applying the updated food preferences control to the preferences profile.

The process of operating a machine learning food preferences algorithm involves aggregating user interactions from linked user services associated with the user profile and a meal plan menu comprising the meal profile, retrieving historic user interactions from a historic interaction database, retrieving similar user interactions from a global user interaction database, and generating an updated food preferences control from the user interactions, the historic user interactions, and the similar user interactions.

In some configurations, the preferences profile comprises food dislikes, food likes, food allergies or restrictions, meal and snack preferences (including preferred recipes), nutrient targets, weight or other personal health objectives, financial budget, preferred food brands, and grocer or food distributor preferences, kcal target and meal presets. The linked user services may comprise a food tracking service and a grocery list service.

Food preference presets may allow a dietitian or nutrition team to group foods together into meals based on the sub categories to which the foods belong. For example, one preset has the following food sub categories for a Sunday breakfast:
Egg product—Egg
Dairy—Yogurt
Beverage—water/tea
Beverage—milk, dairy, 1%
Beverage—Vegetable Juice

Each of these food sub categories contains any number of foods 1-n of which may be brand-specific. The algorithm pulls foods from each food sub category and builds menus for the users based on the user selected presets or by considering user actions.

In some configurations, a user may provide presets defining which food items to utilize for some food sub categories, identify the food item as a meal component and/or part of a larger food component category. The presets may also be utilized to identify specific food items as well as particular combinations of food items that may be viewed as individual meals by themselves.

In some configurations, a user profile may include user preferences such as food preferences (e.g., likes/dislikes, which may be further broken down into preferred tastes and/or textures, smells, etc.), restrictions (e.g., allergies or disease), health objectives (e.g., lose weight), financial budget, grocer or food distributor (e.g., grocer supplier or direct-to-consumer provider in certain food distribution scenarios), preferred brands or private labels, preferred recipes, and preferred restaurants.

The user can add foods that they like, foods they dislike, 1-n preferred brands, recipes, nutrient targets, weight or other personal health objectives, preferred grocer(s) and/or food distributor(s), and kcal target into their preferences before a food menu covering 1-n meals over 1-n days is generated for them.

The algorithm takes the preferences included in the user profile and tailors the food menu with respect to the user's specific needs/wants. The user can also add specific items that they would like to have for every meal. For instance, the user may specify that they would like an apple with every meal or a Dannon yogurt for breakfast and make sure that certain foods are never added to their specific meals.

Presets establish user specified frameworks for determining what foods are included (and excluded) from meals. In addition to establishing global preferences for food and other elements included in the user profile, the user may add increasingly granular information, such as specifying that there is a specific food and/or brand item that they would like to see appear more frequently in the meals generated by the algorithm. This includes the user being able to specify whether they want to eat the specific food item as part of every meal generated by the food menu algorithm, or whether there are specific meals (e.g., breakfast, lunch, dinner, snacks) and which days that they would like to eat those specific food items, or identify that the food can be considered when it can be purchased within financial budget guidelines. Going further, the user can specify 1-n specific macro- and/or micro-nutrient values for specific meals and/or on specific days of a multi-meal menu, for example, an elevated target for sodium or other electrolytes for meals preceding or following a strenuous run.

The food dislikes identify a specific food item and may eliminate it from being selected as a food component of any meal. In some configurations, the user may identify certain times that they would not like to eat certain food items, or when they would not like certain food items suggested. For example, a user may like tuna for lunch or dinner but may specify that they would not like to have tuna added as a food component of a meal for breakfast.

After a menu is generated a number of additional relationships may be added. These relationships pull in entities like the shopping list, food log, fitness log, restaurants, etc.

A food preference optimization algorithm may be incorporated into a meal planning system that allows the system to improve the food suggestions made to users. The algorithm may allow users to import recipes to use as part of their planned meals and then generate suggestions for other recipes based on the combinations of food items in the user's imported recipe(s). The algorithm may optimize food suggestions by identifying similar users utilizing the meal planning system, and utilizing the other user's behaviors such as swapping foods out based on their nutritional components and what foods the other users pair together to develop a taste profile for suggesting combinations of foods.

The algorithm incorporates artificial intelligence elements to enhance the accuracy of the food preference suggestions, including tracking end user behaviors to improve the predictive capability of the algorithm. For example, the algorithm may identify that when 90% of people swap a certain food item, they replace it with another specific food item. The algorithm may determine that the substitution is based on the current context and in the future avoid adding the food item to users with similar taste profiles. Additionally, the algorithm may determine that a substituted food item is highly preferred by certain users with a particular taste profile and make modifications to recipes to include that food item in the future.

In some configurations food preferences may include likes and dislikes as to food type or category, but also including taste, texture, smell, etc.

In some configurations food restrictions may include items to which the user is allergic or which are contraindicated due to a disease or because they conflict with specific nutritional supplements or medications the user is taking.

In some configurations food brands may include products “branded” by a specific food manufacturer, as well as any “private label” or “store brand” or identifier associated with the retailer, and not a supplier.

In some configurations, food distributor may include what we commonly know as a grocery store today, as well as third-party distributors who may provide certain products to grocers, but also, for example, a potential food distribution scenario in the future, where robotic warehouses fill consumer orders that are delivered by driverless vehicles. A “food distributor” could also include restaurants, food delivery services like Blue Apron, etc.

The phrases “in one embodiment”, “in various embodiments”, “in some embodiments”, and the like are used repeatedly. Such phrases do not necessarily refer to the same embodiment. The terms “comprising”, “having”, and “including” are synonymous, unless the context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to or combined, without limiting the scope to the embodiments disclosed herein.

A method of generating geofenced equivalence recommendations for a menu generating system involves communicating the spatiotemporal location, for a user device accessing a user interface (UI) wizard, to a geofenced equivalence recommendations algorithm, configuring the geofenced equivalence recommendations algorithm starting with preferences in the user's profile (including, e.g., food preferences, restrictions, health objectives, budget, preferred brands, grocers or food distributors, and kcal target), and adding user location preferences comprising a food source type parameter, a food source distance parameter, and proximal food preferences received from the UI wizard, identifying a geographic region and a geolocation from the spatiotemporal location through operation of the geofenced equivalence recommendations algorithm, configuring a food component selector with the geographic region, the geolocation, and the user location preferences to identify relevant proximal food databases, and configuring a menu generation algorithm with the geographic region, the geolocation, the proximal food preferences, and user meal preferences to generate at least one spatiotemporal location based meal entry in a meal plan menu from the relevant proximal food databases.

In some configurations, the geofenced equivalence recommendations algorithm may be initially configured with user meal preferences from in the user's profile (including, e.g., food preferences, restrictions, health objectives, budget, preferred brands, grocers or food distributors, and kcal target), as a source for initial configurations.

In some configurations, the user meal preferences comprise food dislikes, food likes, food allergies or restrictions, meal and snack preferences (including preferred recipes), nutrient targets, weight or other personal health objectives, preferred food brands, and grocer or food distributor preferences, kcal target and meal presets. The linked user services may comprise a food tracking service and a grocery list service.

In some configurations, a user may provide presets defining which food items to utilize for some food sub categories, identify the food item as a meal component and/or part of a larger food component category. The presets may also be utilized to identify specific food items as well as particular combinations of food items that may be viewed as individual meals by themselves.

In some configurations, a user profile may include user preferences such as food preferences (e.g., likes/dislikes, which may be further broken down into preferred tastes and/or textures, smells, etc.), restrictions (e.g., allergies or disease), health objectives (e.g., lose weight), financial budget, grocer or food distributor (e.g., grocer supplier or direct-to-consumer provider in certain food distribution scenarios), preferred brands or private labels, preferred recipes, and preferred restaurants.

In some configurations, the method of generating geofenced equivalence recommendations for a menu generating system may also include receiving the geolocation of the mobile device through the mobile device's location services at the geofenced equivalence recommendations algorithm, and identifying the geographic region from the geolocation through operation of the geofenced equivalence recommendations algorithm.

Furthermore, in some instances, the method of generating geofenced equivalence recommendations for a menu generating system may receive the spatiotemporal location from a user's calendar service linked with the menu generating system.

In some configurations, the method of generating geofenced equivalence recommendations for a meal plan may utilize a set up assistant (wizard) to configure a geofenced equivalence recommendations algorithm to configure a meal plan generation algorithm to create/update a meal, a 1-n meal plan for 1-n days, and shopping lists derived therefrom, based on a user's travel.

A user wanting to receive location based meal plan menus may operate a geofenced equivalence recommendations wizard with the meal plan generation application on the user's mobile device. Once the geofenced equivalence recommendations wizard is open on the user's mobile device, the wizard displays a few questions to the user to configure the geofenced equivalence recommendations algorithm. The wizard requests information regarding the type of food options source from which a user wishes to obtain food (e.g., prepared meals options (e.g., restaurants, delivery, etc.,) or food components (e.g., grocery stores)), the distance the user is willing to travel to obtain the foods whiles they are traveling, and the proximal food preferences and food brands a user may have available while they are traveling (e.g., traveling to Philadelphia, a user may want to have a cheesesteak, or only want to have Dannon Yogurt purchased at a Safeway). The wizard takes the user responses and configures the meal plan generation application to display all the matching restaurants, grocery stores, and other food or meal access options in the geo tagged area to where the user will be traveling and/or the user's user device location. The user selects a day/meal range identifying the days/times that the user will be in the particular area, and then the meal plan generation algorithm generates the needed items and updates the menu. In some configurations, the system may allow food vendors to read information from the planned menu and allow the venders to set up delivery times or have meals ready for the user to pick up.

The wizard may also provide the user a number of food options to select from for that travel location allowing the user to select the best meal based on the preferences of the user and the available menu options.

The meal plan generation application may allow the user to select whatever meal they want from a restaurant's menu. If the user is selecting either a breakfast, morning snack, lunch, or afternoon snack, then the meal plan generation algorithm will re-calculate the dinner/evening meal/snack selections to get closer to hitting the nutrient and kcal targets for that day.

The meal plan generation application may also generate the best three options for the user based on the preferences the user has in their profile. All three choices may result in the previously generated meal plan menu being updated such as, having the appropriate meals swapped out, recalculating future meals, etc.

One of skill in the art will realize that the methods and apparatuses of this disclosure describe prescribed functionality associated with a specific, structured graphical interface. Specifically, the methods and apparatuses, inter alia, are directed to a system and method for generating geofenced equivalence recommendations for a menu generating system. One of skill in the art will realize that these methods are significantly more than abstract data collection and manipulation.

Further, the methods provide a technological solution to a technological problem, and do not merely state the outcome or results of the solution. As an example, the system and method for generating geofenced equivalence recommendations for a menu generating system displays an updated food menu that accounts for a user's caloric and nutritional goals, and other relevant preferences, to a user through a user interface, based in part on the user's travel plans or their device location. Furthermore, the system and method for generating geofenced equivalence recommendations for a menu generating system reduces the load on the system by requiring fewer inputs from the user in creating the updated food menu, freeing system resources and thus improving the efficiency of the user interfacing device. This is a particular technological solution producing a technological and tangible result. The methods are directed to a specific technique that improves the relevant technology and are not merely a result or effect.

Additionally, the methods produce the useful, concrete, and tangible result of the system and method for generating geofenced equivalence recommendations for a menu generating system, thereby identifying each change as associated with its antecedent rule set.

Further, the methods are directed to a specifically-structured graphical user interface, where the structure is coupled to specific functionality. More specifically, the methods disclose a specific set of information to the user, rather than using conventional user interface methods to display a generic index on a computer.

A system and method for modifying dietary related behavior provides users with personalized coaching to encourage a user to make healthier life choices based on their dietary and fitness related goals. The system utilizes a machine learning algorithm that incorporates behavioral studies from various research sources to create and modify contact with a user that is more likely to result in the desired change in their behavior. The system may provide the user with a behavior modifying notification following the detection of a target behavior or action by the user. The behavior modifying notification may encourage a user to continue performing the detected behavior or advise the user of the risk if they continue that behavior. The system may additionally incorporate information from a wearable or carried device such as a smart health device, to improve the accuracy of the behavior modifying notification. The system may also communicate with a meal plan generation system to identify a user's food preferences and dietary goals.

A method of operating a system for modifying behavior involves generating behavior adherence data from monitored behavior data, meal planning data, meal consumption (or food log) data, and planned activities data through operation of a behavior analyzer. Behavior adherence data is stored as historical user behavior in a controlled memory data structure. A behavior modifying notification is generated from demographic information, the behavior adherence data, the historical user behavior, health and behavior research data, biometric data, and location data from a user's mobile device, through operation of a machine learning algorithm. The behavior modifying notification is displayed through a display device of the mobile device, and the displayed behavior modifying notification is communicated to the behavior analyzer for generating the behavior adherence data.

The method of operating the system for modifying behavior may additionally include a smart health device to provide the biometric data to the machine learning algorithm. In the method of operating the system for modifying behavior, the monitored behavior data comprises physical activity data and user food log data.

In the method of operating the system for modifying behavior, the physical activity data may be provided by a smart health device. In the method of operating the system for modifying behavior, the meal planning data may be provided by a meal plan generation system. In the method of operating the system for modifying behavior, the meal planning data may comprise an intake targets and goals and a proposed meal plan.

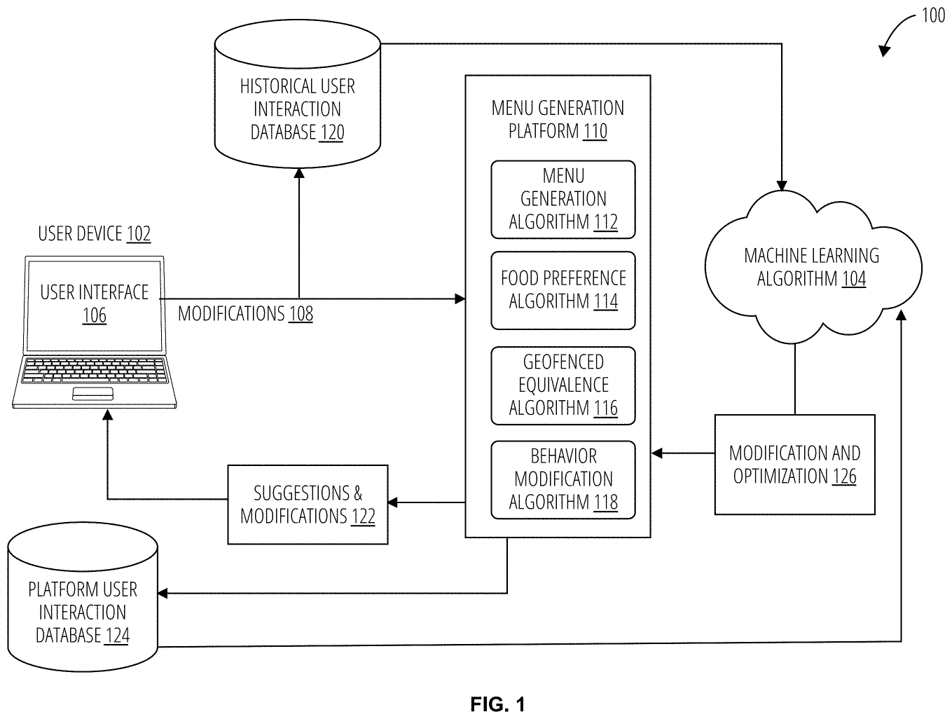

Referencing FIG. 1, a system 100 comprises a user device 102, a historical user interaction database 120, a menu generation platform 110, a machine learning algorithm 104, and a platform user interaction database 124. The user device 102 comprises a user interface 106 that communicates modifications 108 to the menu generation platform 110. The modifications 108 are aggregated by the historical user interaction database 120 and provided to the machine learning algorithm 104. The menu generation platform 110 comprises a menu generation algorithm 112, a food preference algorithm 114, a geofenced equivalence recommendations algorithm 116, and a behavior modification algorithm 118. The menu generation algorithm 112, the food preference algorithm 114, the geofenced equivalence recommendations algorithm 116, and the behavior modification algorithm 118 are operations that improve the user experience by reducing the number of user inputs required to provide a meal plan menu for at least one day. The menu generation platform 110 may aggregate user interactions from all users utilizing its platform in a platform user interaction database 124. The machine learning algorithm 104 utilizes the entries stored in the historical user interaction database 120 and the platform user interaction database 124 to generate modification and optimization controls 126 for the menu generation platform 110. The menu generation platform 110 may utilize the modification and optimization controls 126 to generate suggestions & modifications 122 for the menu generation algorithm 112, the food preference algorithm 114, the geofenced equivalence recommendations algorithm 116, and the behavior modification algorithm 118 displayable through a user interface 106 of the user device 102.

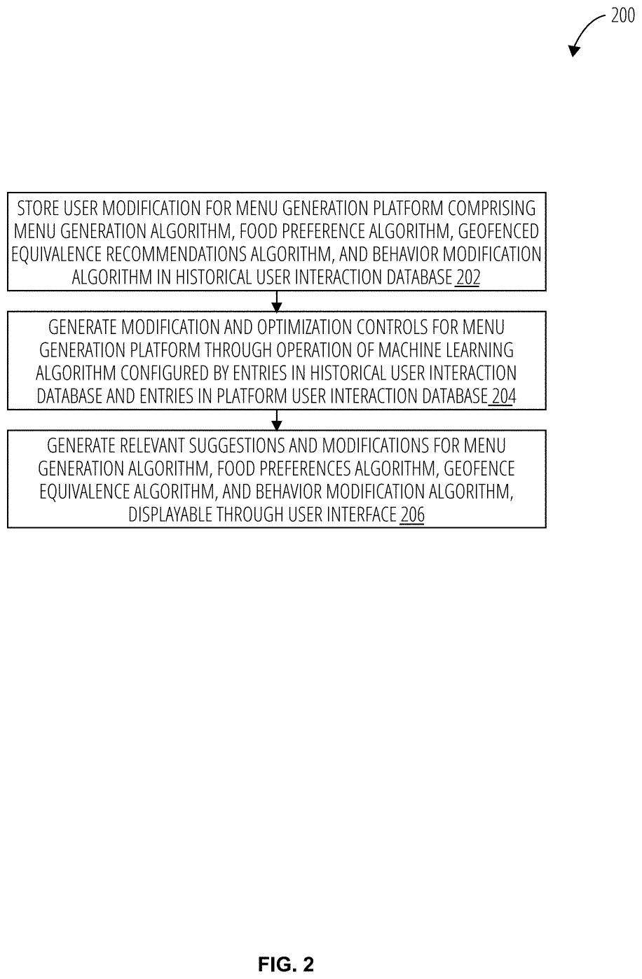

Referencing FIG. 2, a method 200 stores user modification for a menu generation platform comprising a menu generation algorithm, a food preference algorithm, a geofenced equivalence recommendations algorithm, and a behavior modification algorithm in a historical user interaction database (block 202). In block 204, method 200 generates modification and optimization controls for the menu generation platform through operation of the machine learning algorithm configured by entries in the historical user interaction database and entries in a platform user interaction database. In block 206, method 200 generates relevant suggestions and modifications for the menu generation algorithm, the food preferences algorithm, geofence equivalence algorithm, and behavior modification algorithm, displayable through a user interface.

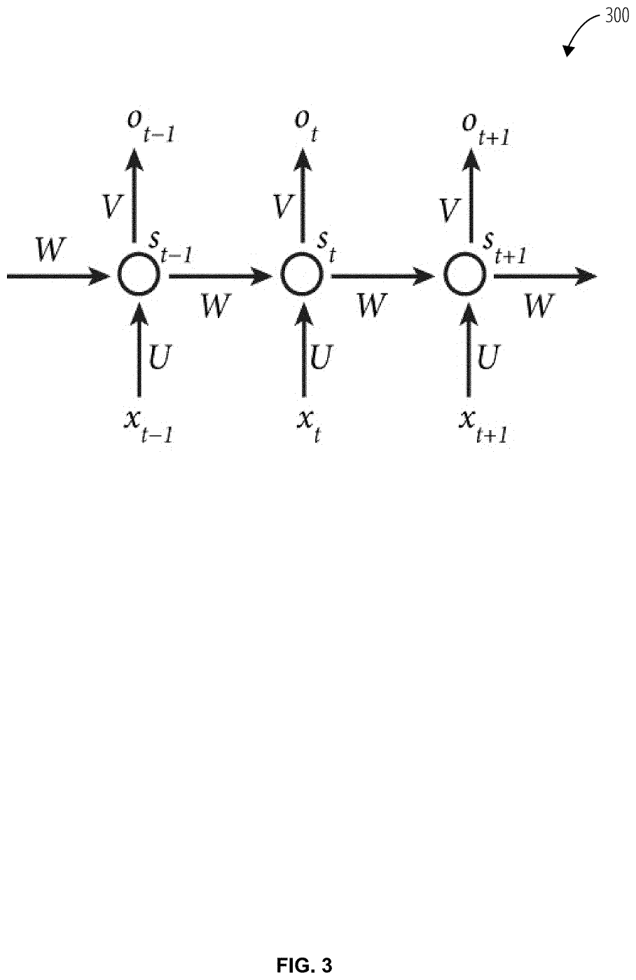

FIG. 3 illustrates a recurrent neural network 300 (RNN). Variable x[t] is the input at stage t. For example, x[1] could be a one-hot vector corresponding to the second word of a sentence. Variable s[t] is the hidden state at stage t. It's the “memory” of the network. The variable s[t] is calculated based on the previous hidden state and the input at the current stage: s[t]=f(Ux[t]+Ws[t-1]). The activation function f usually is a nonlinearity such as tanh or ReLU. The variable s(−1) , which is required to calculate the first hidden state, is typically initialized to all zeroes. Variable o[t] is the output at stage t. For example, to predict the next word in a sentence it would be a vector of probabilities across the vocabulary: o[t]=softmax(Vs[t]) .

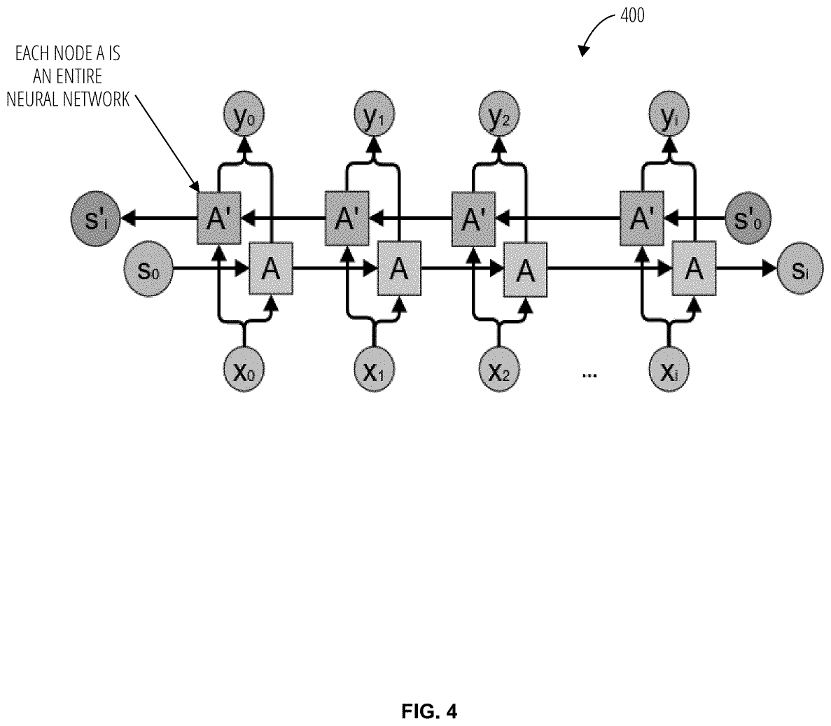

FIG. 4 illustrates a bidirectional recurrent neural network 400 (BRNN). BRNNs are based on the idea that the output at a stage may not only depend on the previous inputs in the sequence, but also future elements. For example, to predict a missing word in a sequence a BRNN will consider both the left and the right context. BRNNs may be implemented as two RNNs in which the output is computed based on the hidden state of both RNNs.

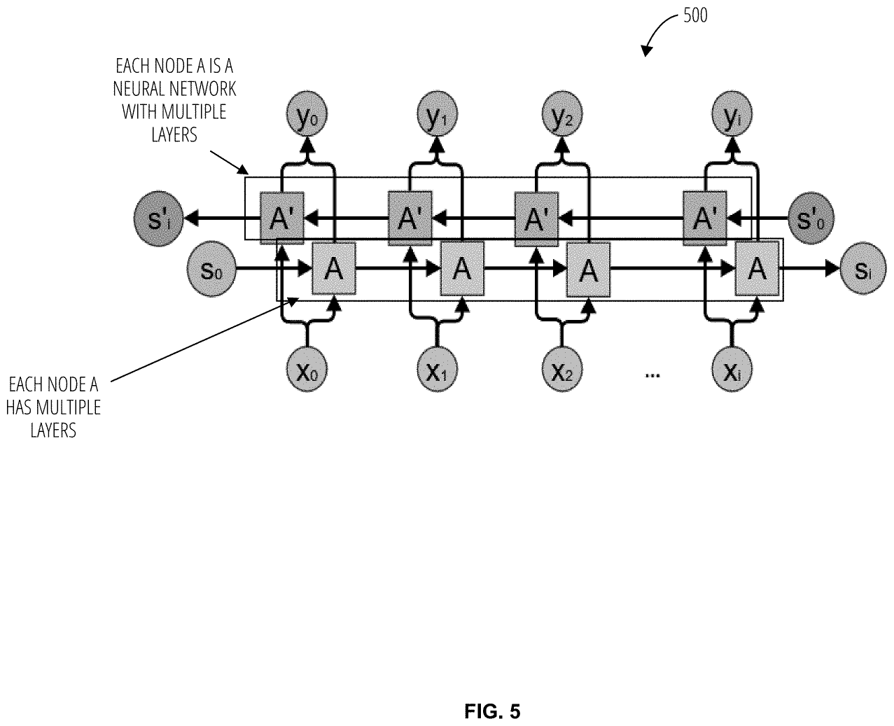

FIG. 5 illustrates a deep bidirectional recurrent neural network 500. Deep BRNNs are similar to BRNNs, but multiple layers per stage. In practice this enables a higher learning capacity but also requires more training data than for single layer networks.

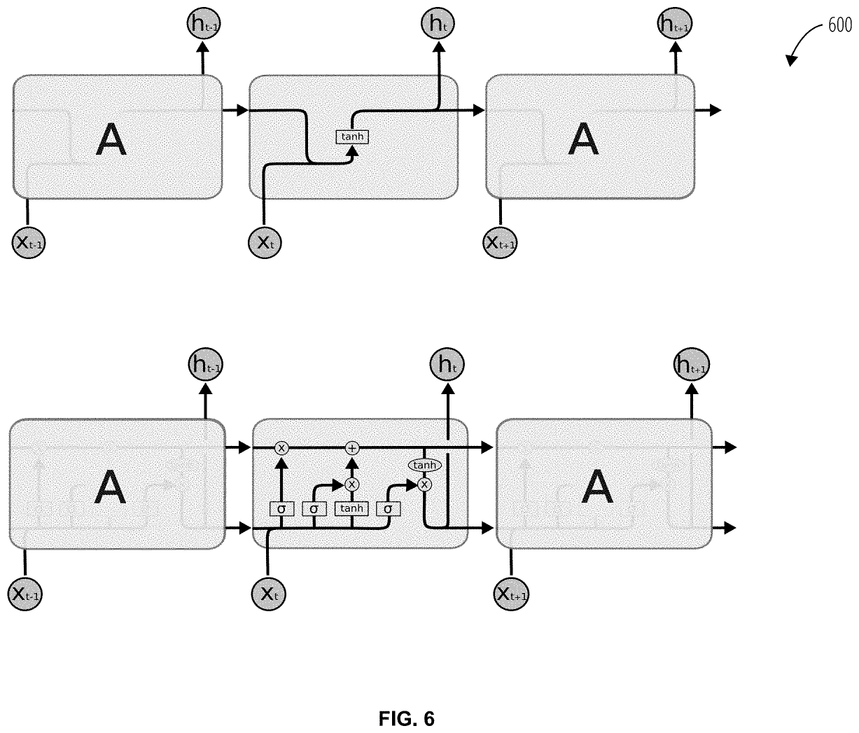

FIG. 6 illustrates a RNN architecture with long short-term memory 600 (LSTM).

All RNNs have the form of a chain of repeating nodes, each node being a neural network. In standard RNNs, this repeating node will have a structure such as a single tanh layer. This is shown in the upper diagram.

An LSTMs (lower diagram) also has this chain like design, but the repeating node has a different structure than for regular RNNs. Instead of having a single neural network layer, there are typically four, and the layers interact in a particular way.

In an LSTM each path carries an entire vector, from the output of one node to the inputs of others. The pink circles represent pointwise operations, like vector addition, while the yellow boxes are learned neural network layers. Lines merging denote concatenation, while a line forking denote values being copied and the copies going to different locations.

An important feature of LSTMs is the cell state Ct, the horizontal line running through the top of the long short-term memory 600.

The cell state is like a conveyor belt. It runs across the entire chain, with only some minor linear interactions. It's entirely possible for signals to flow along it unchanged.

The LSTM has the ability to remove or add information to the cell state, carefully regulated by structures called gates. Gates are a way to optionally let information through a cell. They are typically formed using a sigmoid neural net layer and a pointwise multiplication operation.

The sigmoid layer outputs numbers between zero and one, describing how much of each component should be let through. A value of zero means “let nothing through,” while a value of one means “let everything through”

An LSTM has three of these sigmoid gates, to protect and control the cell state.

A basic deep neural network 700 is based on a collection of connected units or nodes called artificial neurons which loosely model the neurons in a biological brain. Each connection, like the synapses in a biological brain, can transmit a signal from one artificial neuron to another. An artificial neuron that receives a signal can process it and then signal additional artificial neurons connected to it.

In common implementations, the signal at a connection between artificial neurons is a real number, and the output of each artificial neuron is computed by some non-linear function (the activation function) of the sum of its inputs. The connections between artificial neurons are called ‘edges’ or axons. Artificial neurons and edges typically have a weight that adjusts as learning proceeds. The weight increases or decreases the strength of the signal at a connection. Artificial neurons may have a threshold (trigger threshold) such that the signal is only sent if the aggregate signal crosses that threshold. Typically, artificial neurons are aggregated into layers. Different layers may perform different kinds of transformations on their inputs. Signals travel from the first layer (the input layer), to the last layer (the output layer), possibly after traversing one or more intermediate (hidden) layers.

A neuron with label receiving inputs from predecessor neurons consists of the following components:
an activation input that depends on a time or index parameter;
an optional threshold, which stays fixed unless changed by a learning function;
an activation function that computes the activation output at a given time or index from the previous neuron inputs, activation, and threshold if any; and
and an optional output function computing the output from the activation.

An input neuron has no predecessor but serves as input interface for the whole network. Similarly, an output neuron has no successor and thus serves as output interface of the whole network.

The network includes connections, each connection transferring the output of a neuron in one layer to the input of a neuron in a next layer. Each connection is assigned a weight.

The propagation function computes the input to the neuron from the outputs of predecessor neurons and typically has the form of a sum of products of the weighted values on the outputs of the predecessor neurons.

The learning rule is a rule or an algorithm which modifies the parameters of the neural network, in order for a given input to the network to produce a favored output. This learning process typically involves modifying the weights and thresholds of the neurons and axons within the network.

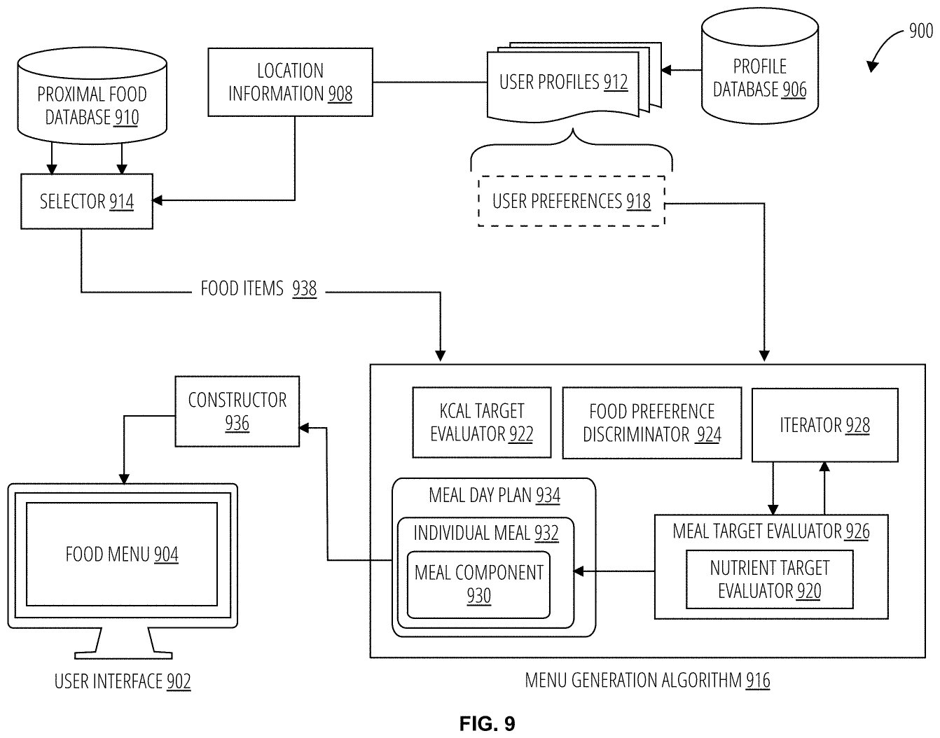

Referencing FIG. 9, a system 900 for operating a meal plan optimization algorithm includes a user interface 902, a profile database 906, a selector 914, a menu generation algorithm 916, and constructor 936. The profile database 906 comprises at least one user profile 912 each comprising location information 908 and user preferences 918. The location information 908 configures the selector 914, identifying at least one proximal food database to select food items 938 to generate the food menu for at least one day 904. The menu generation algorithm 916 receives the user preferences 918 from the at least one user profile 912 and configures a kcal target evaluator 922, a food preference discriminator 924, and a meal target evaluator 926. The food preference discriminator 924 is configured to identify the food items 938 to use as meal component 930 for an individual meal 932. The menu generation algorithm 916 sets preferred food items for an individual meal 932. The meal target evaluator 926 utilizes the user preferences 918 to configure the nutrient target evaluator 920 to identify food item substitutions and portion size adjustments that best fit individual meals. With individual meals identified for a meal day plan 934, the menu generation algorithm 916 communicates the meal day plans to the constructor 936 which modifies the kcal target evaluator 922 to generate a food menu for at least one day 904 to be displayed through the user interface 902.

In some configurations, the user profile comprises user preferences comprising food preferences (i.e., likes/dislikes), food restrictions (e.g., gluten free), health objectives (e.g., lose weight), budget, preferred brands and/or private labels, and preferred grocers and/or food distributors.

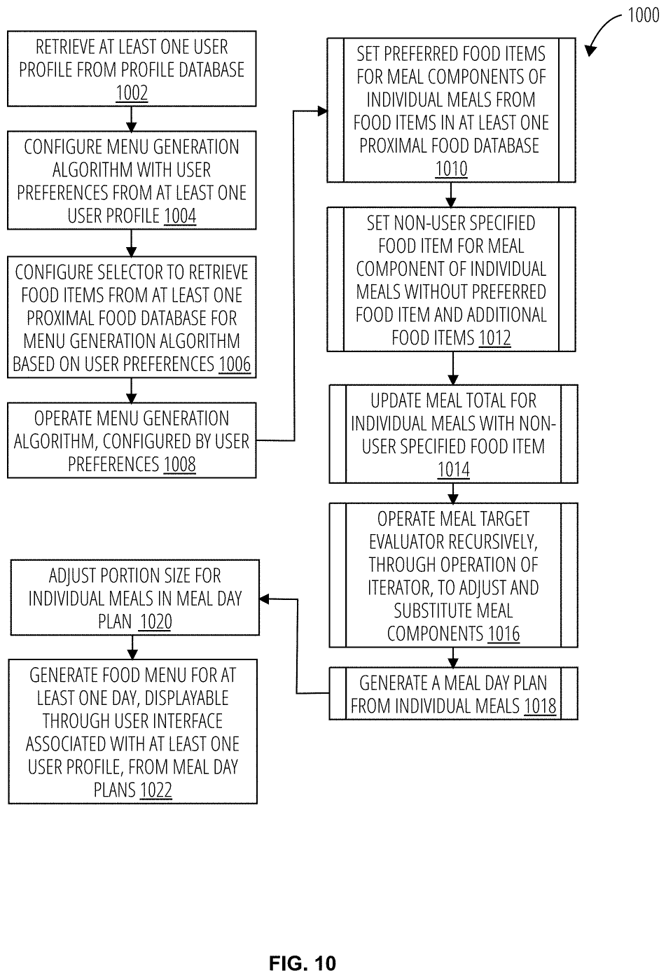

The system 900 may be operated in accordance with the processes described in FIG. 10.

Referencing FIG. 10, a method 1000 for operating a meal plan nutrition optimization algorithm involves retrieving at least one user profile from a profile database (block 1002). In block 1004, the method 1000 configures a menu generation algorithm with user preferences from the at least one user profile. In block 1006, the method 1000 configures a selector to retrieve food items from at least one proximal food database for the menu generation algorithm, from the user preferences. In block 1008, the method 1000 operates the menu generation algorithm, configured by the user preferences. In subroutine block 1010, the menu generation algorithm sets preferred food items for meal components of individual meals from the food items in the at least one proximal food database through operation of a food preference discriminator. In subroutine block 1012, the menu generation algorithm sets a non-user specified food item for a meal component of the individual meals without a preferred food item and additional food items through operation of the food preference discriminator. In subroutine block 1014, the menu generation algorithm updates a meal total for the individual meals with the non-user specified food item. In subroutine block 1016, the menu generation algorithm operates a meal target evaluator recursively, through operation of an iterator, to adjust and substitute meal components. In subroutine block 1018, the menu generation algorithm generates a meal day plan from the individual meals. In block 1020, the method 1000 adjusts the portion size for the individual meals in the meal day plan through operation of the kcal target evaluator, configured by the user preferences. In block 1022, the method 1000 generates a food menu for at least one day, displayable through a user interface associated with the at least one user profile, from meal day plans through operation of a constructor.

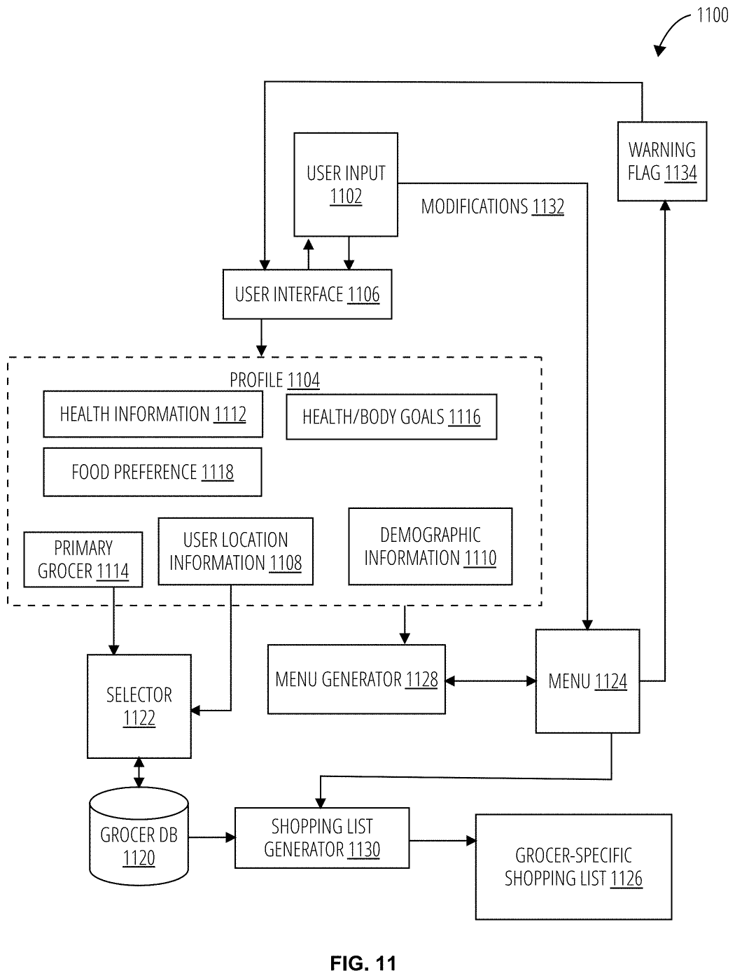

FIG. 11 illustrates an example system 1100 that comprises a profile 1104 that includes health information 1112, food preference 1118, health/body goals 1116, primary grocer 1114, user location information 1108, and demographic information 1110. The profile 1104 is initially configured through a user interface 1106 receiving user input 1102. A selector 1122 is configured utilizing the primary grocer 1114 and user location information 1108 to select a local grocer database 1120. The menu generation algorithm 1128 is configured utilizing the profile 1104 to generate a menu 1124. The local grocer database 1120 and the menu 1124 are utilized by a shopping list generator 1130 to generate a grocer-specific shopping list 1126. A warning flag 1134 may be detected in the menu 1124 that may be presented to the user through a user interface 1106. The user may address the warning flag 1134 in the menu 1124 by entering modifications 1132 to the menu 1124. In some configurations, the warning flag 1134 may be activated variously, including an immediate warning if a user enters a food item that is inconsistent with a user's food restrictions or is contraindicated based on nutritional supplements or medications taken by the user, or after a meal plan has been generated, to indicate, for example, that a meal exceeds the kcal target. The warning can be communicated in various ways, including in one embodiment a red, yellow, green system that indicates in a readily understandable way, whether a meal or food item is potentially dangerous (red), worthy of more consideration by the user (yellow), or “all clear” (green).

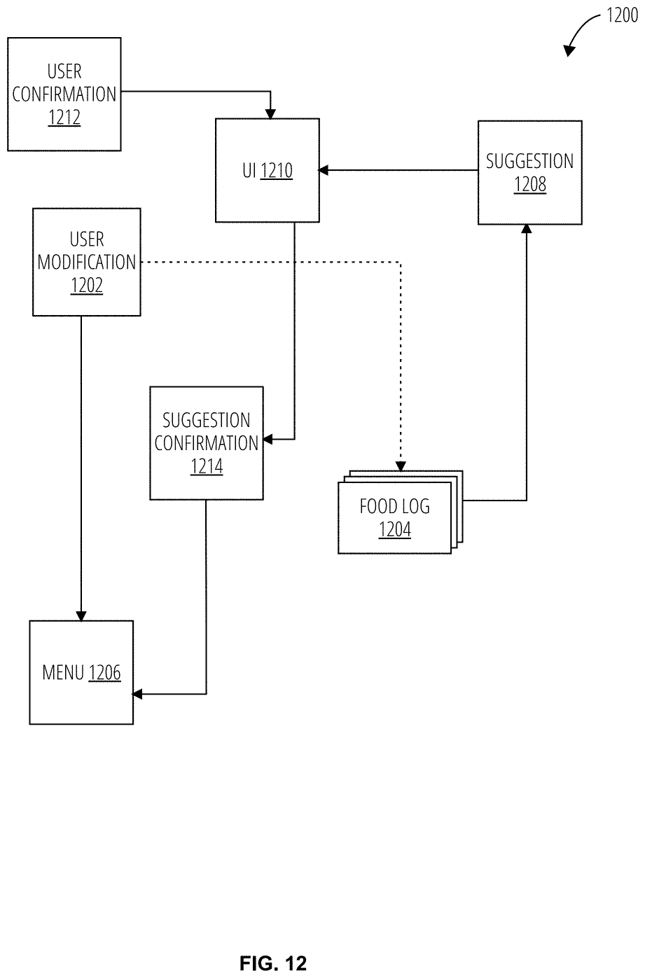

FIG. 12 illustrates an example system 1200 that shows the interactions between a UI 1210 that receives a suggestion 1208 and a user confirmation 1212 to generate a suggestion confirmation 1214 regarding food items within a menu 1206. The user modification 1202 may change food items within the menu 1206 which may also be utilized to populate a food log 1204. The food log 1204 may allow the system 1200 to provide an improved suggestion 1208 to the user through UI 1210.

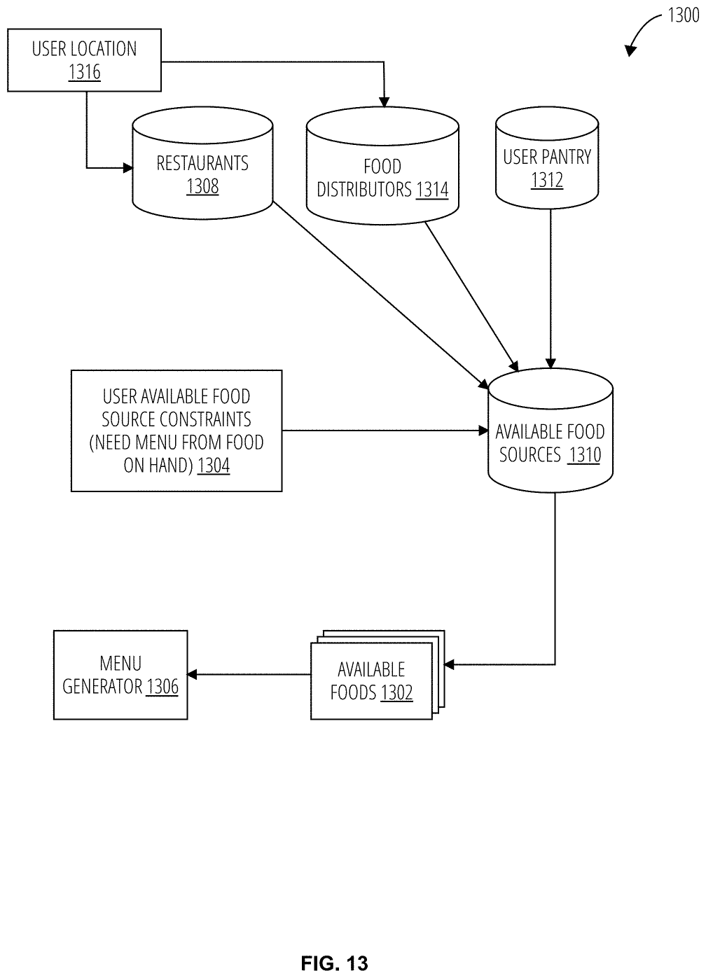

FIG. 13 illustrates an example system 1300 that utilizes user location 1316 to determine the restaurants 1308 and the food distributors 1314 nearby the user as available food sources 1310 for generating meals. The system 1300 may also utilize the contents within the user pantry 1312 as available food sources 1310. In some instances, user available food source constraints (need menu from food on hand) 1304 may require a user to modify their meals on their menus based on the available food sources 1310 dependent on the user location 1316. The available food sources 1310 may be identified as available foods 1302 from which the menu generator 1306 may select to generate or modify the meal plan menu.

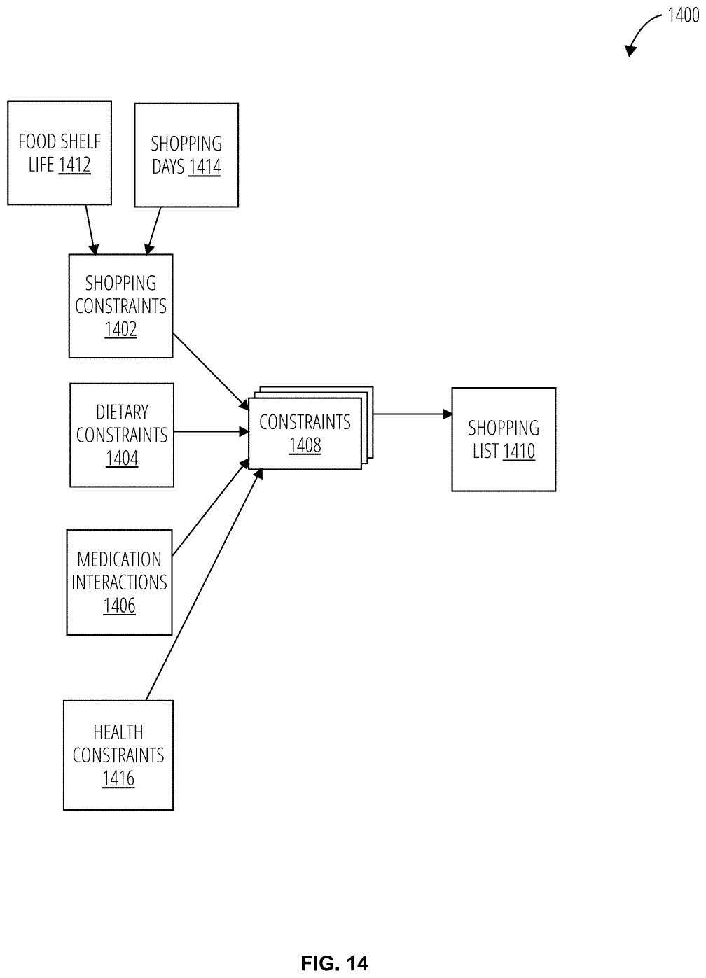

FIG. 14 illustrates an example system 1400 that identifies constraints 1408 that may be utilized in the generation of a shopping list 1410. The constraints 1408 includes shopping constraints 1402 which may also include the food shelf life 1412 and shopping days 1414 available to the user, dietary constraints 1404, medication interactions 1406, and health constraints 1416. In some configurations, the constraints 1408 may include other limitations chosen by the user and/or imposed on the user.

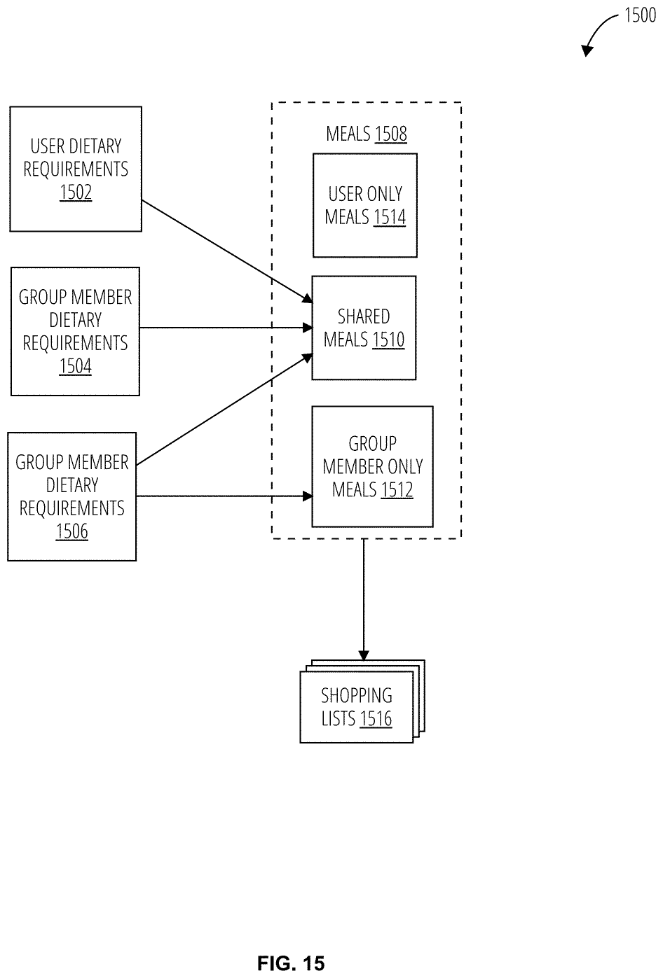

FIG. 15 illustrates an example system 1500 showing the influencers for different categories of meals based on shared profiles. The meals 1508 include user only meals 1514, shared meals 1510, and group member only meals 1512. Group member only meals 1512 may be any meal another member of the group eats that does not include other members of the group. When determining meals for multiple individuals in a group, the user dietary requirements 1502 influence the shared meals 1510, which may also be influenced by the group member dietary requirements 1504 and the group member dietary requirements 1506 of the other members of a family. For the other members of the group, their group member dietary requirements 1506 may also affect group member only meals 1512 that only the other member(s) eat. The meals 1508 from all group members are then utilized to generate a shopping lists 1516.

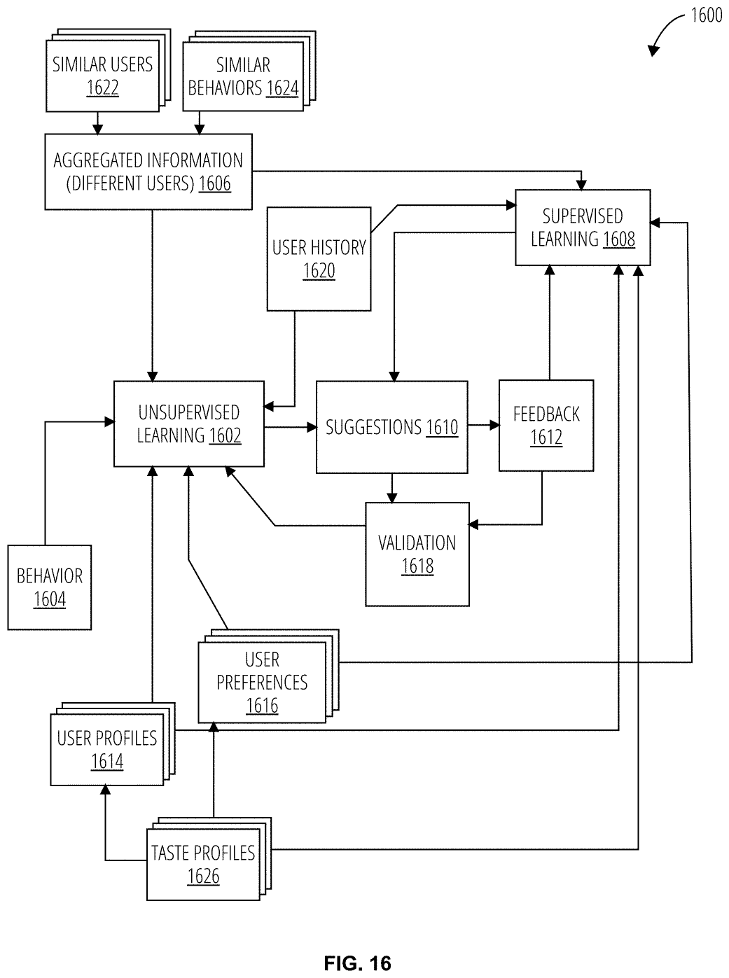

Referencing FIG. 16, a system 1600 illustrates a behavior learning process in which aggregated information (different users) 1606, user data, may be collected from similar users 1622 with similar behaviors 1624 and runs an unsupervised learning process 1602 and a supervised learning process 1608. The unsupervised learning process 1602 incorporates behavior 1604, user profiles 1614, user preferences 1616, and user history 1620 to generate suggestions 1610. The suggestions 1610 may be provided with feedback 1612, which serves as validation 1618 for the unsupervised learning process 1602. The supervised learning process 1608 may incorporate the user preferences 1616, user profiles 1614, and user history 1620 to generate suggestions 1610. The suggestions 1610 may be provided with feedback 1612, which helps improve the supervised learning process 1608. The user profiles 1614 and the user preferences 1616 may also include taste profiles 1626 utilized by either the unsupervised learning process 1602 or the supervised learning process 1608 for generating suggestions 1610.

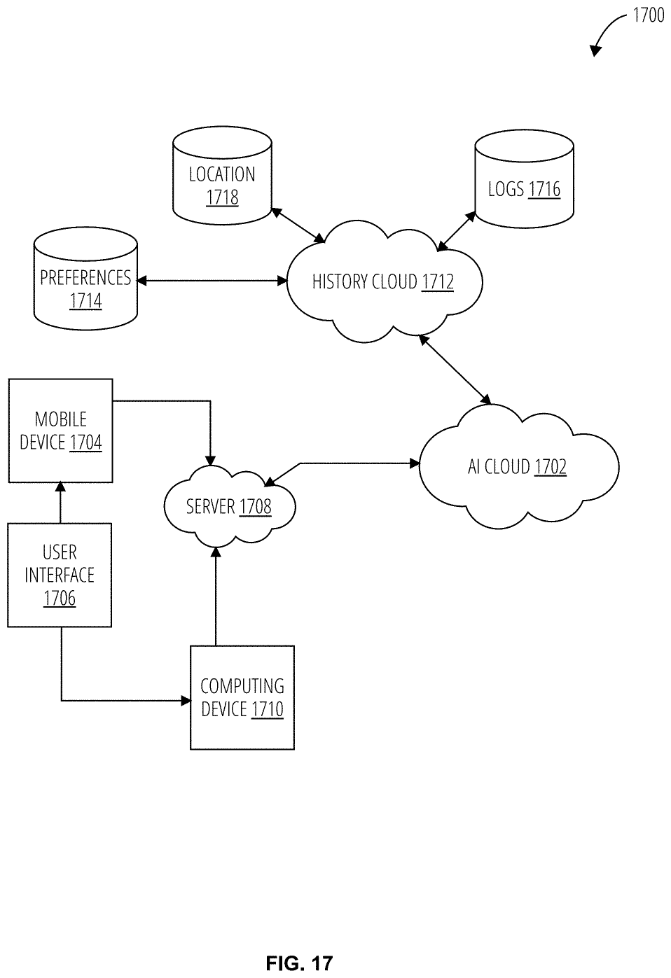

Referencing FIG. 17, a system 1700 involves a history cloud 1712 that bidirectionally communicates with system logs 1716, location 1718, and preferences 1714 storage. A server 1708 may receive user interactions via a user interface 1706 by way of a mobile device 1704 or a computing device 1710. The server 1708 may communicate the user information to an artificial intelligence (AI) cloud 1102, which may correlate the user interactions received from the server to information stored on the history cloud 1712.

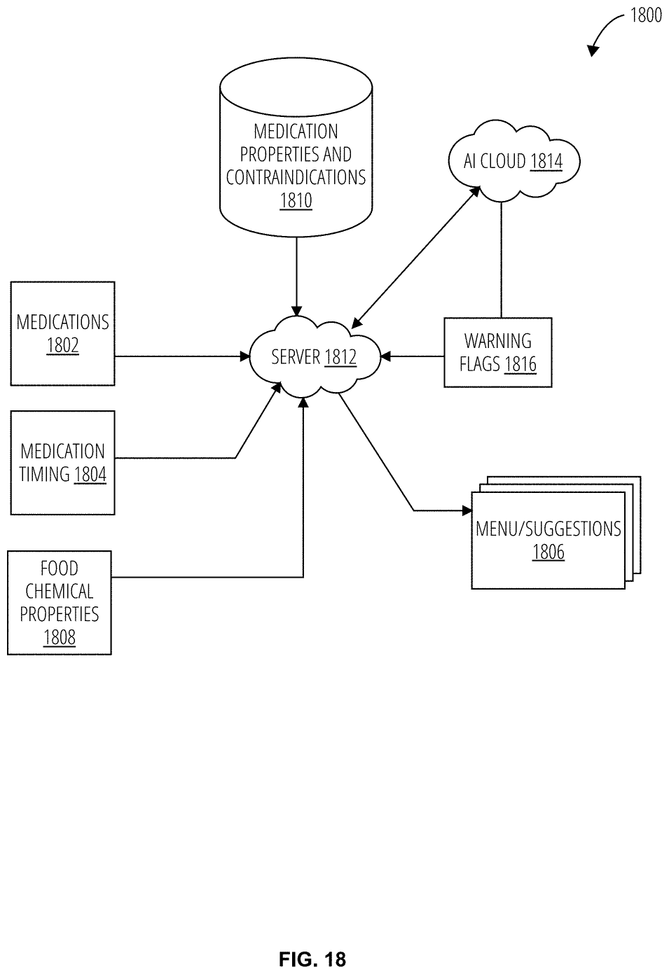

Referencing FIG. 18, a system 1800 illustrates how a server 1812 receives information regarding medications 1802, medication timing 1804, and food chemical properties 1808, and references those inputs with a database containing medication properties and contraindications 1810. The server 1812 may also provide the information to an AI cloud 1814 which may help in the generation of a menu or food suggestions 1206.

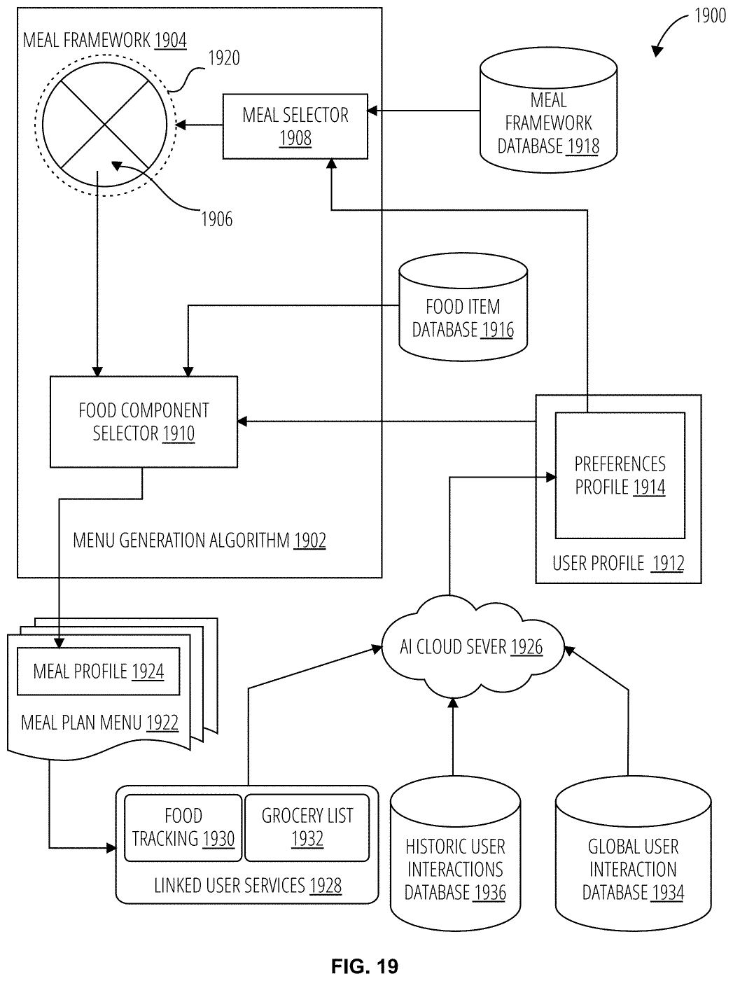

Referencing FIG. 19, a system 1900 includes a menu generation algorithm 1902, a meal framework database 1918, a food item database 1916, a linked user services 1928, a historic user interactions database 1936, a global user interaction database 1934, and a user profile 1912. The user profile 1912 comprises a preferences profile 1914. In some configurations, the preferences profile may include meal presets, food likes, food dislikes, food restrictions, health objectives, financial budget, brand preferences, and grocer and/or food distributor preferences. The menu generation algorithm 1902 comprises a meal selector 1908 and a food component selector 1910. The preferences profile 1914 configures a meal selector 1908 to retrieve a meal framework 1904 from the meal framework database 1918. The selected framework meal 1920 comprises at least one food component category 1906. The preferences profile 1914 and the at least one food component category 1906 are utilized to configure a food component selector 1910 to retrieve food items corresponding to the food component category 1906. The food component selector 1910 utilizes the food items to generate a meal profile 1924 as part of a meal plan menu 1922. The machine learning food preferences algorithm (AI cloud server) 1926 receives information from the linked user services 1928 associated with the meal plan menu 1922 and the user profile 1912, a historic user interactions database 1936, and a global user interaction database 1934. In some configurations, the linked user services 1928 comprises services such as a food tracking service 1930 and a grocery list service 1932. The machine learning food preferences algorithm (AI cloud server) 1926 utilizes the information to modify the preferences profile 1914.

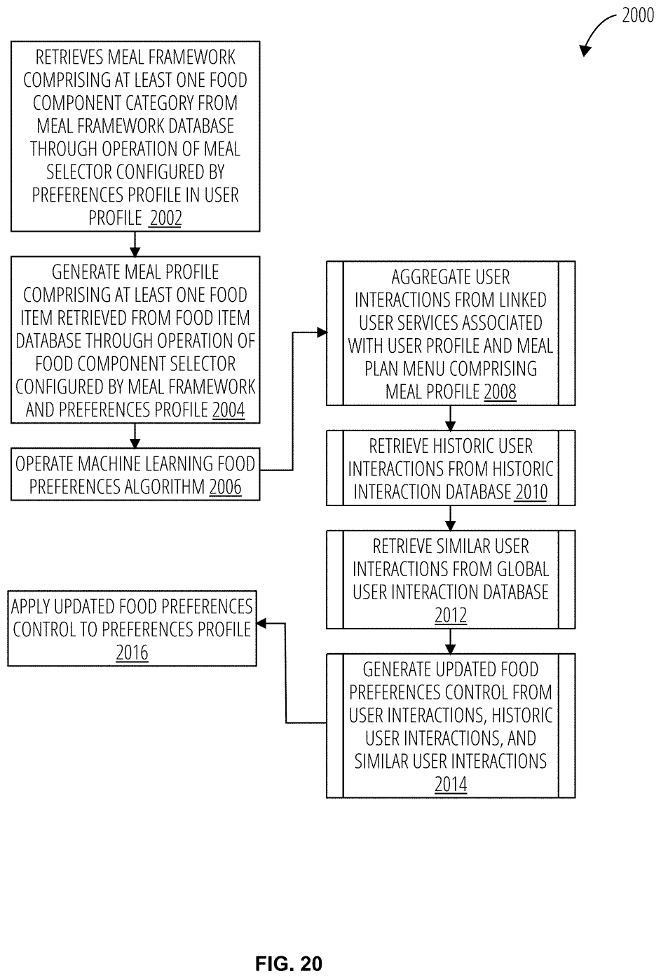

Referencing FIG. 20, a method 2000 for operating a food preference algorithm includes retrieving a meal framework comprising at least one food component category from a meal framework database through operation of a meal selector configured by a preferences profile in a user profile (block 2002). In block 2004, the method 2000 generates a meal profile comprising at least one food item retrieved from a food item database through operation of a food component selector configured by the meal framework and the preferences profile. In block 2006, the method 2000 operates a machine learning food preferences algorithm. In subroutine block 2008, the machine learning food preferences algorithm aggregates user interactions from linked user services associated with the user profile and a meal plan menu comprising the meal profile. In subroutine block 2010, the machine learning food preferences algorithm retrieves historic user interactions from a historic interaction database. In subroutine block 2012, the machine learning food preferences algorithm retrieves similar user interactions from a global user interaction database. In subroutine block 2014, the machine learning food preferences algorithm generates an updated food preferences control from the user interactions, the historic user interactions, and the similar user interactions. In block 2016, the method 2000 applies the updated food preferences control to the preferences profile.

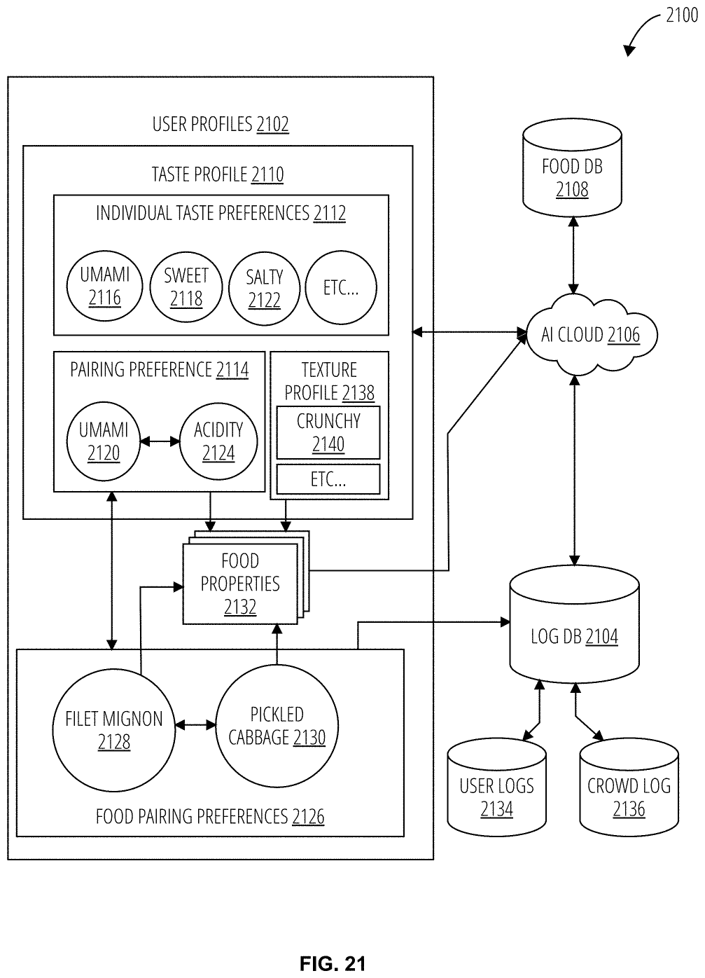

Referencing FIG. 21, a system 2100 comprises user profiles 2102, a log database 2104, a machine learning food preferences algorithm (AI cloud) 2106, and a food item database 2108. The user profiles 2102 comprise a taste profile 2110 and food pairing preferences 2126. The taste profile 2110 may comprise individual taste preferences 2112 including taste values for commonly accepted tastes that include, but are not limited to, umami 2116, sweet 2118, and salty 2122. The taste profile 2110 may also include pairing preference 2114 including flavor pairing preference values for umami 2120 and acidity 2124. The taste profile 2110 may also include a texture profile 2138 that include, for example, a preference for crunchy 2140. The food texture profile is referenced with respect to each food item, and user-specified texture or “mouth feel” preferences, determined by user input or as revealed by user activity, and is combined with the food taste profile to enhance the accuracy of the food selections included in user meals. The pairing preference 2114 may be utilized in combination with established food pairing preferences 2126 that may include, for example, a flavor combination for filet mignon 2128 and pickled cabbage 2130. The log database 2104 includes information for user logs 2134 and global user logs 2136. The food pairing preferences 2126 may record a user's food pairing preferences 2126 in the user logs 2134. The machine learning food preferences algorithm (AI cloud) 2106 utilizes food properties 2132 that are received from the food pairing preferences 2126 and the pairing preference 2114, the food items in the food item database 2108, and logs in the log database 2104 to more accurately describe the taste profile 2110.

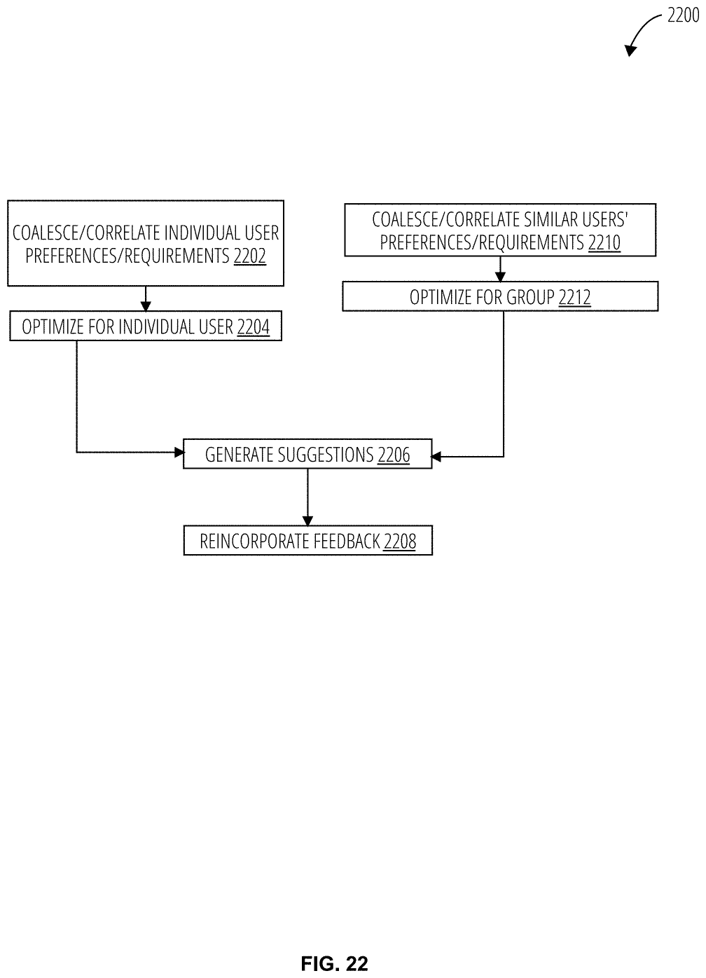

Referencing FIG. 22, a process 2200 involves coalescing/correlating individual user preferences/requirements for their dietary needs (block 2202). In block 2204, the process 2200 optimizes the food preferences for an individual user. The process 2200 includes an additional branch running parallel with the branch starting with block 2202. This additional branch begins with block 2210, where the process 2200 coalesces/correlates similar users' food preferences/requirements. The branch then continues to block 2212, where the process 2200 optimize food preferences for the group of similar users. Both branches meet at block 2206, where the process 2200 generates food item suggestions. In block 2208, the process 2200 reincorporates feedback to improve food item suggestions.

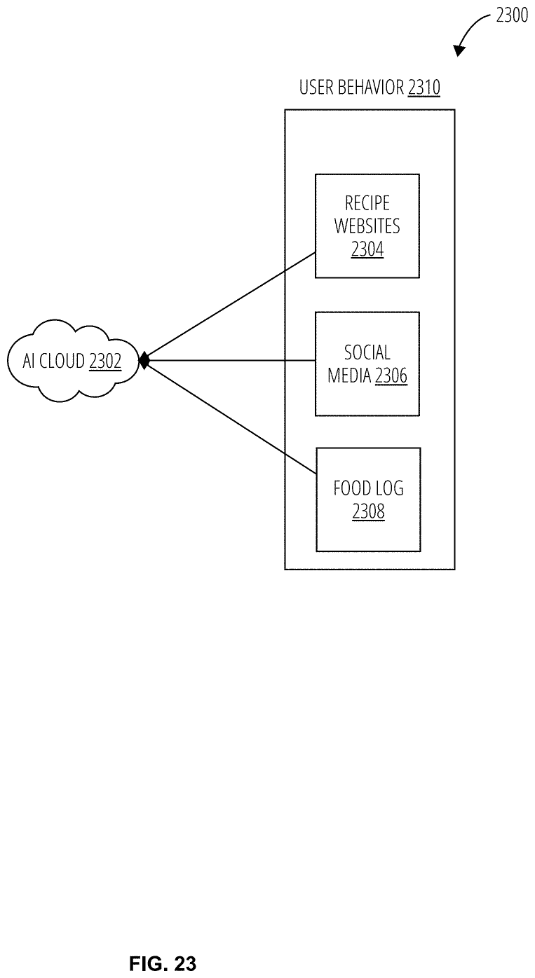

Referencing FIG. 23, a system 2300 comprises a machine learning food preferences algorithm (AI cloud) 2302 that utilizes user behavior 2310 comprising recipe websites 2304, social media 2306, and a food log 2308 associated with a user to improve user food preferences.

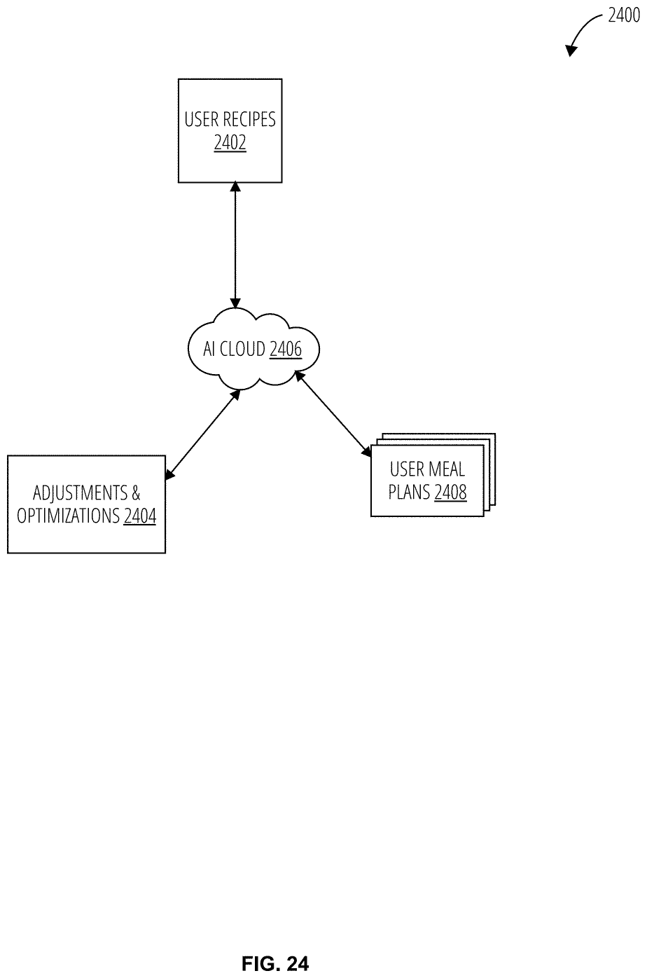

Referencing FIG. 24, a system 2400 comprises a machine learning food preferences algorithm (AI cloud) 2406 that utilizes user recipes 2402, user meal plans 2408, and user adjustments & optimizations 2404 to improve user food preferences.

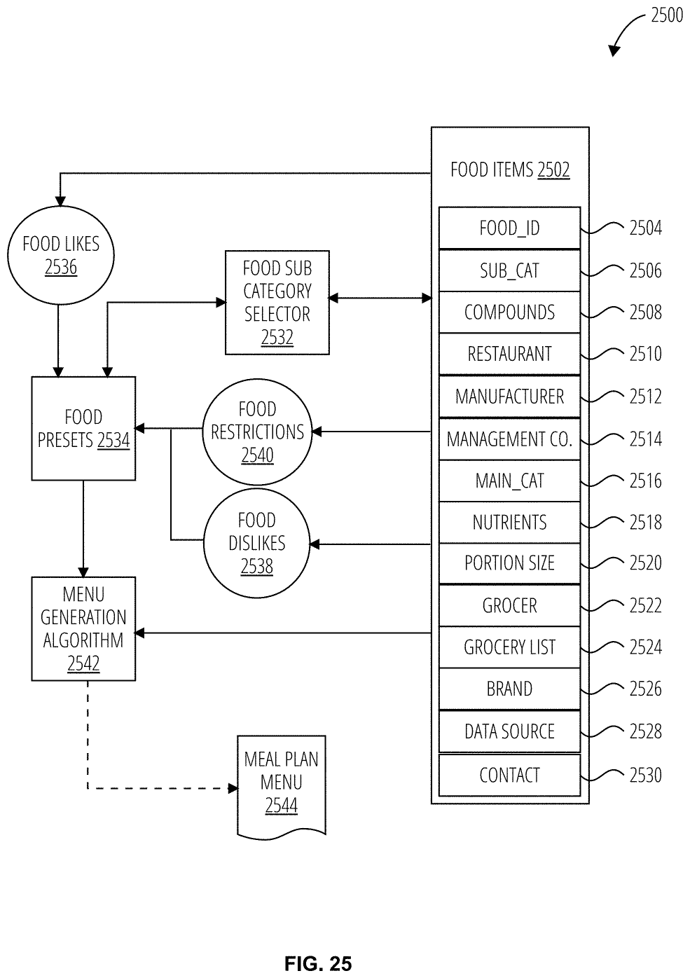

Referencing FIG. 25 a system 2500 includes food items 2502 comprising a food item identifier 2504, a food component sub category 2506, a relevant food compounds 708, a restaurant identifier 2510, a food manufacturer 2512, a food management company 714, a main food category 2516, nutrient quantities 2518, a portion size 2520, a specific food distributor/grocer location 2522, related grocery list information 2524, a food brand name 2526, a food data source 2528, and food manufacturer contact information 2530. A food sub category selector 2532 identifies food items 2502 in relevant food component sub categories based on food presets 2534. The food presets 2534 utilize the food restrictions 2540, food dislikes 2538, and the food likes 2536 configured by a user to further filter food items 2502 utilized in the generation of a meal plan menu 2544 by the menu generation algorithm 2542.

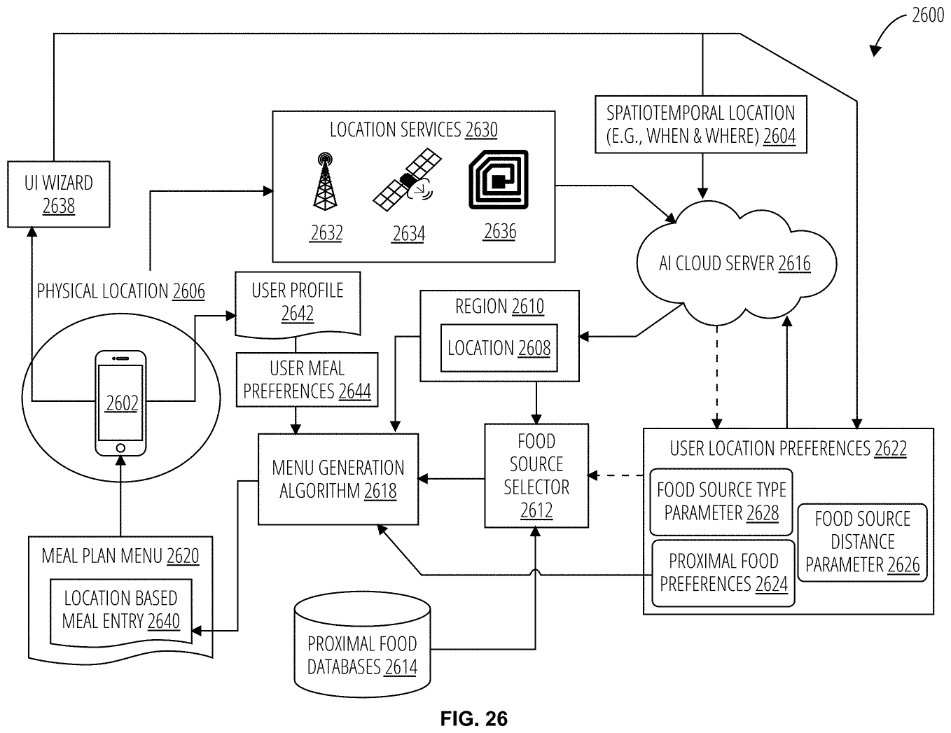

Referencing FIG. 26, a system 2600 comprises a UI wizard 2638 operated on a user device 2602, a geofenced equivalence recommendations algorithm (AI cloud server) 2616, a food source selector 2612, a menu generation algorithm 2618, and proximal food databases 2614. The user device 2602 shares a spatiotemporal location 2604 and user location preferences 2622 to a geofenced equivalence recommendations algorithm (AI cloud server) 2616 operating on an AI cloud server through a UI wizard 2638 operating with the menu generation system.

The geofenced equivalence recommendations algorithm (AI cloud server) 2616 utilizes the spatiotemporal location 2604 and the user location preferences 2622 comprising a food source type parameter 2628, a proximal food preferences 2624, and a food source distance parameter 2626, to identify a geographic region 2610 and a geolocation 2608 where the user device 2602 will be. The food source selector 2612 utilizes the geographic region 2610, the geolocation 2608, the proximal food preferences 2624, the food source type parameter 2628, and the food source distance parameter 2626, to identify relevant local food sources from proximal food databases 2614. The menu generation algorithm 2618 is configured by a user meal preferences 2644 from a user profile 2642 associated with the user device 2602, the geographic region 2610, and the proximal food preferences 2624 to select meals or food components from the relevant proximal food databases identified by the food source selector 2612 in order to generate an at least one spatiotemporal location based meal entry 2640 as part of a meal plan menu 2620. The meal plan menu 2620 is display to the user device 2602. In some configurations, the user device 2602 may provide its physical location 2606 to the geofenced equivalence recommendations algorithm (AI cloud server) 2616 through location services 2630 running on the user device 2602. The location services 2630 may include, but are not limited to, location data provided by a wireless network 2632 (e.g., cell towers, Wi-Fi, global positioning system (gps 2634) data, and location information data from near field location beacons 2636 (e.g., Bluetooth beacons, near field communication (NFC) beacons, etc., and location information captured by means of scanning or other visual data capture (e.g., scan of a QR code posted at a particular location). In some configurations, the user meal preferences 2644 may comprise food dislikes, food likes, food allergies or restrictions, meal and snack preferences (including preferred recipes), nutrient targets, weight or other personal health objectives, preferred food brands, and grocer or food distributor preferences, kcal target and meal presets. The linked user services may comprise a food tracking service and a grocery list service.

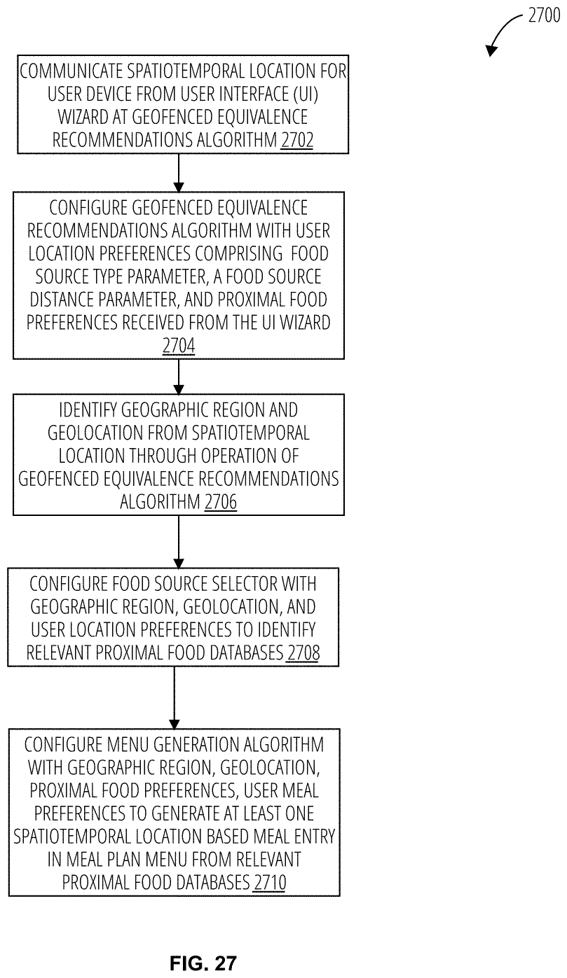

The system 2600 may be operated in accordance with the process described in FIG. 27.

Referencing FIG. 27, a method 2700 for generating geofenced equivalence recommendations for a menu generating system involves communicating spatiotemporal location from a user device accessing a user interface (UI) wizard at a geofenced equivalence recommendations algorithm (block 2702). In block 2704, method 2700 configures the geofenced equivalence recommendations algorithm with user location preferences comprising a food source type parameter, a food source distance parameter, and proximal food preferences received from the UI wizard. In block 2706, method 2700 identifies a geographic region and a geolocation from the spatiotemporal location through operation of the geofenced equivalence recommendations algorithm. In block 2708, method 2700 configures a food source selector with the geographic region, the geolocation, and the user location preferences to identify relevant proximal food databases. In block 2710, method 2700 configures a menu generation algorithm with the geographic region, the geolocation, the proximal food preferences, and user meal preferences to generate at least one spatiotemporal location based meal entry in a meal plan menu from the relevant proximal food databases.

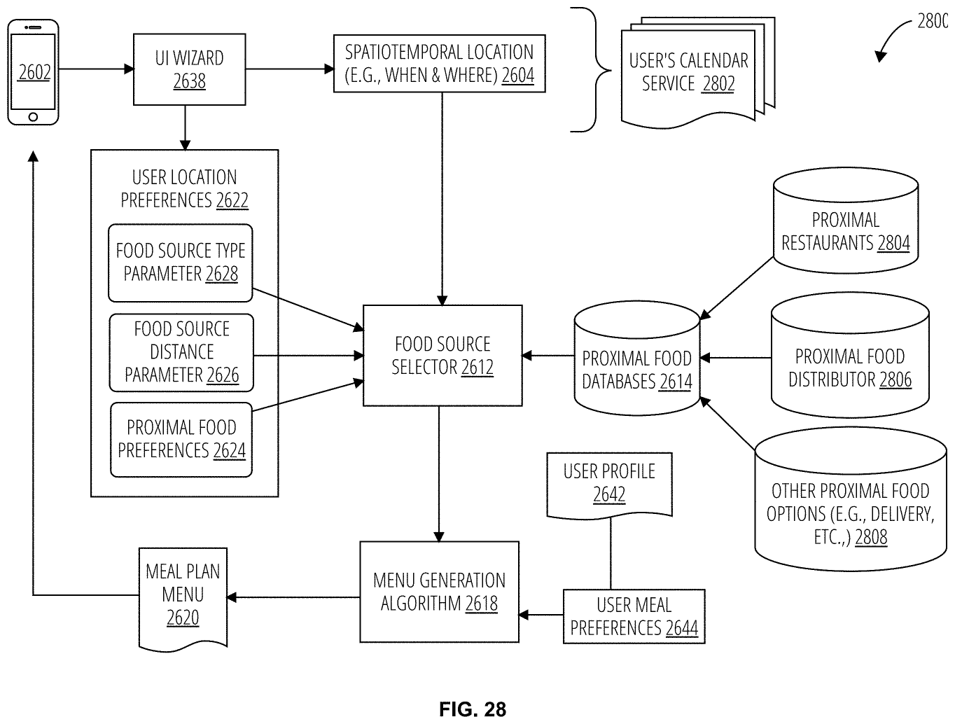

Referencing FIG. 28, a system 2800 illustrates a geofenced equivalence recommendations algorithm receiving a spatiotemporal location 2604 from a user device 2602 by way of the UI wizard 2638. The UI wizard 2638 also provides the user location preferences 2622 to configure the food source selector 2612 to select relevant proximal food databases from a proximal food databases 2614 comprising a proximal restaurant database 2804, a proximal food distributor database 2806, and an other local food options other local food options database 2808 (e.g., brands). The system 2800 may also receive the spatiotemporal location 2604 from a user's calendar service 2802. The food source selector 2612 utilizes the food source type parameter 2628, the food source distance parameter 2626, and the proximal food preferences 2624 to provide a menu generation algorithm 2618 with the relevant proximal food databases and the user meal preferences 2644 from the user profile 2642 to generate a meal plan menu 2620.

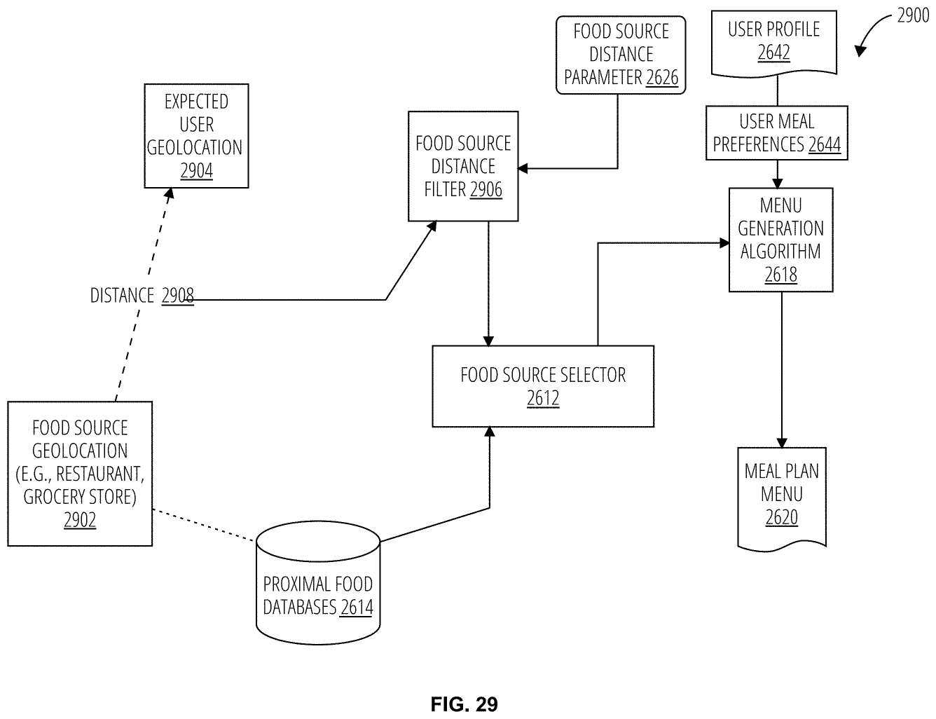

Referencing FIG. 29, a system 2900 illustrates the selection of a local food source database from the proximal food databases 2614 based on the distance 2908 between the food source geolocation 2902 and the expected user geolocation 2904 through the use of a food source distance filter 2906 configured by the food source distance parameter 2626. The food source distance filter 2906 configures the food source selector 2612 to select local food source databases where the distance 2908 is within the food source distance parameter 2626 in order to provide those databases to the menu generation algorithm 2618 to generate a meal plan menu 2620.

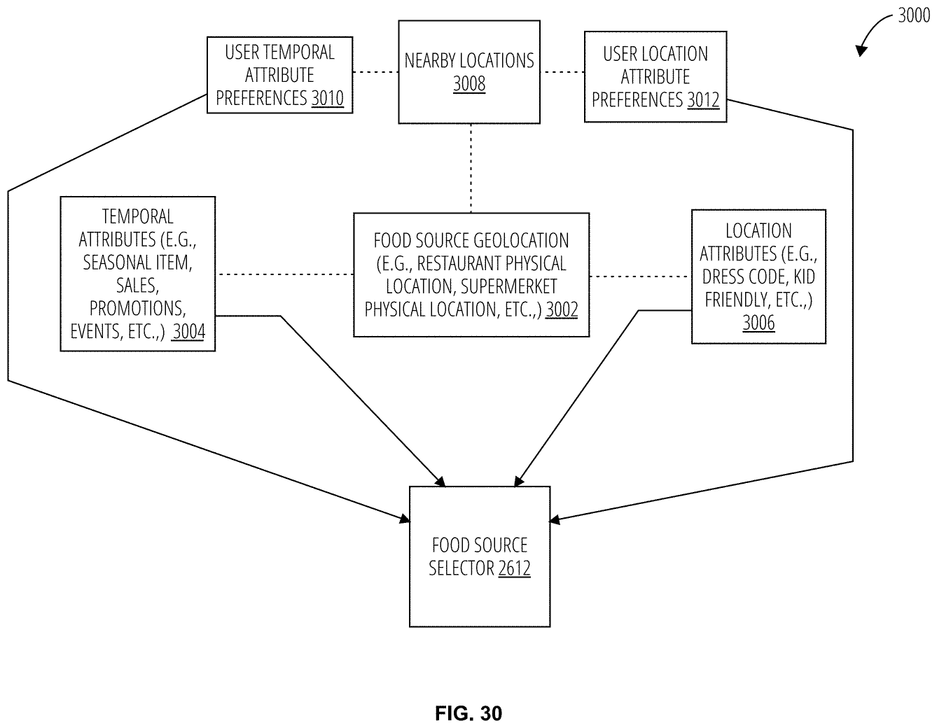

Referencing FIG. 30, a system 3000 illustrates a configuration where in addition to filtering a food source geolocation 3002 based on distance, (e.g., if it is a nearby locations 3008) the food source geolocation 3002 may be filtered based on user temporal attribute preferences 3010 and user location attribute preferences 3012 associated with it. For example, the food source geolocation 3002 comprises temporal attributes 3004 (e.g., seasonal item, sales, promotions, events, etc.,) and location attributes 3006 (e.g., dress code, kid friendly, etc.,), a user may select to avoid locations that fall outside of their preferences. The user temporal attribute preferences 3010 and the user location attribute preferences 3012 may be utilized by the food source selector 2612 do select from among relevant proximal food databases.

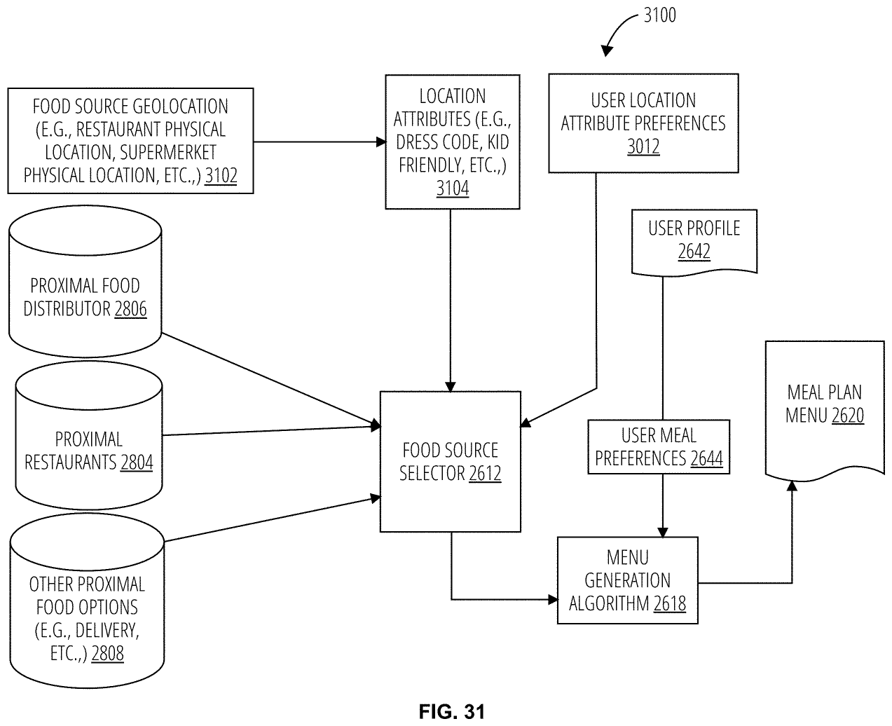

Referencing FIG. 31, a system 3100 illustrates how the geofenced equivalence recommendations algorithm communicates the food source location attributes 3104 of a food source geolocation 3102 to help the food source selector 2612 to select the relevant proximal food databases based on the user location attribute preferences 3012.

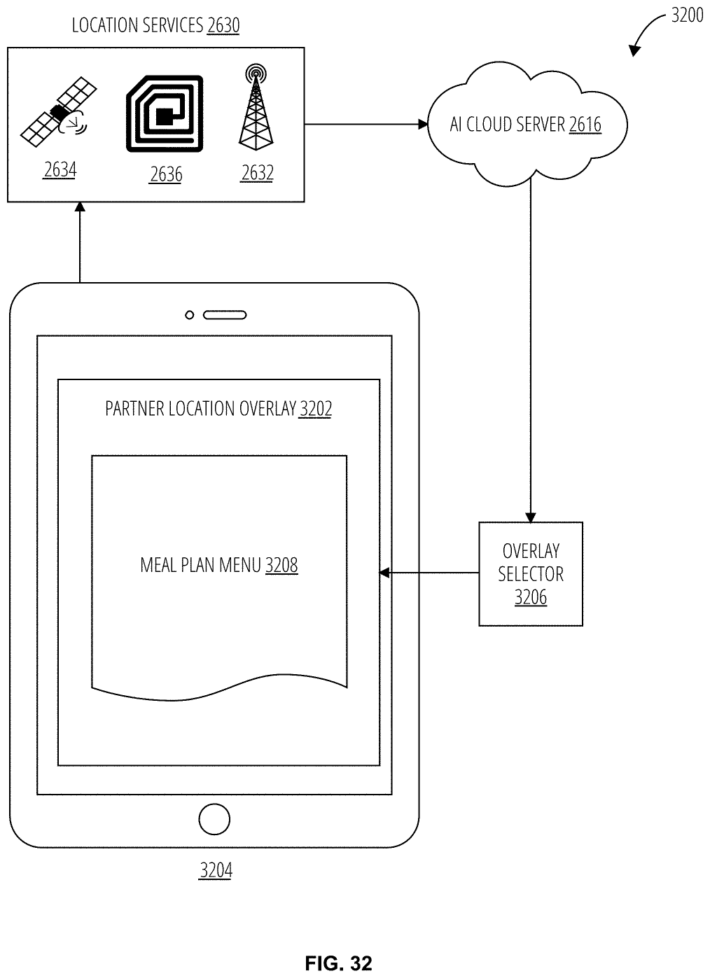

Referencing FIG. 32, a system 3200 illustrates a user device 3204 displaying a partner location overlay 3202 with the meal plan menu 3208 within a user interface. The partner location overlay 3202 may be selected using an overlay selector 3206 configured by the geofenced equivalence recommendations algorithm (AI cloud server) 2616 based on the geolocation of the user device 3204 provided by the location services 2630.

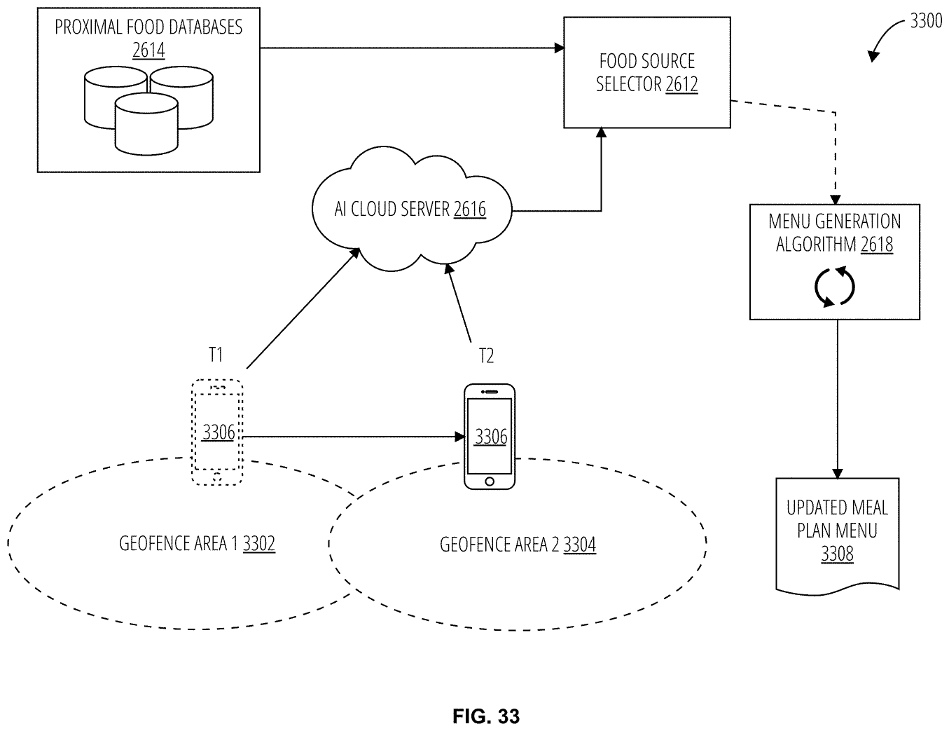

Referencing FIG. 33, a system 3300 illustrates the process of generating an updated meal plan menu 3308 based on the movement of the user's user device. At T1 the user device 3306 is in geofence area 1 3302 and communicates this information to the geofenced equivalence recommendations algorithm (AI cloud server) 2616. When the user device 3306 moves into geofence area 2 3304 at T2, the geofenced equivalence recommendations algorithm (AI cloud server) 2616 detects the change in the geofenced area and communicates the information to the food source selector 2612 to select relevant proximal food databases for the menu generation algorithm 2618. The menu generation algorithm 2618 then generates an updated meal plan menu 3308 with updated location information.

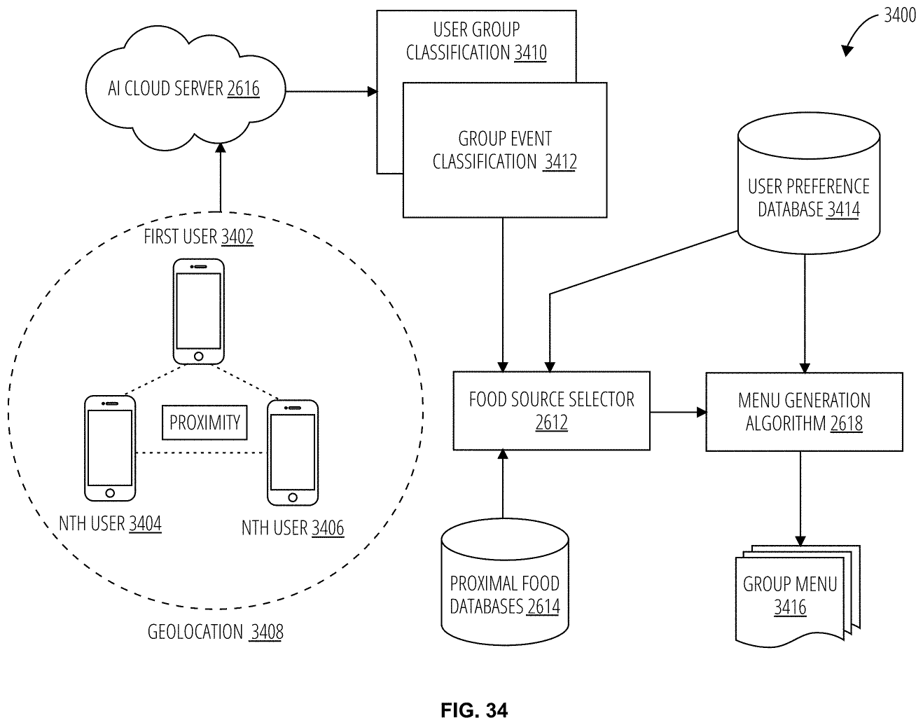

Referencing FIG. 34, a system 3400 illustrates a configuration where a group menu 3416 may be generated for a set of users based on their proximity to each other within a geolocation 3408. For example, based on proximity, a geofenced equivalence recommendations algorithm (AI cloud server) 2616 may determine that a first user 3402, an nth user 3404, and nth user 3406, are in the same geolocation 3408. The geofenced equivalence recommendations algorithm (AI cloud server) 2616 may then identify a user group classification 3410 to determine if an existing relationship exists between the users and if so determine if there is a group event classification 3412. Based on the user group classification 3410, and/or the group event classification 3412, the user preferences from a user preference database 3414, the food source selector 2612 may select relevant proximal food databases to all the users to generate a group menu 3416.

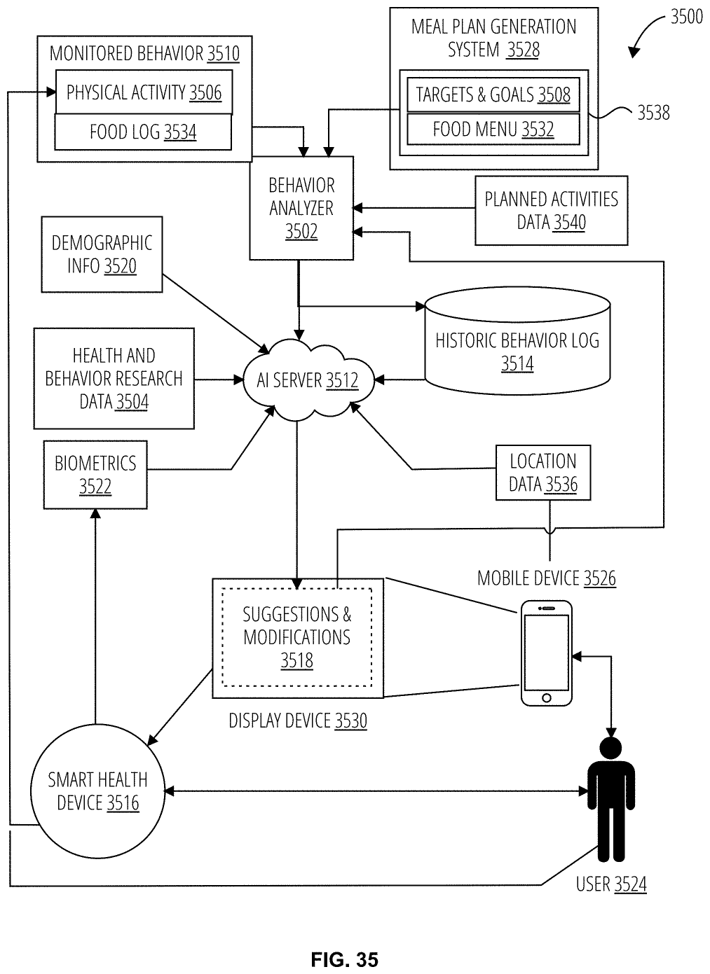

Referencing FIG. 35, a system 3500 includes a meal plan generation system 3528, a behavior analyzer 3502, a machine learning algorithm 3512 (AI server), a mobile device 3526, and a smart health device 3516. The behavior analyzer 3502 collects monitored behavior data 3510 comprising physical activity data 3506 and a user food log data 3534, meal planning data 3538 comprising intake targets and goals 108 and proposed meal plan 3532 (food menu), and planned activities data 3540 and generates behavior adherence data. The meal planning data 3538 may be provided by a meal plan generation system 3528 utilized to assist a user in generating a meal plan for a future period of time. The behavior adherence data is stored as historical user behavior 3514 in a controlled memory data structure and is provided to the machine learning algorithm 3512 (AI server) for generating a behavior modifying notification 3518 displayable in a display device 3530. In some configurations, the behavior modifying notification 3518 may include device configurations (e.g., trigger conditions) to deliver an alert to the user in response to a series of actions and events. For example, the smart health device 3516 may function as a blood glucose and ketone monitor that detects when levels are at certain range defined by the machine learning algorithm, to notify the user through a vibration on their smart health device on through an alert displayable on the user device. The machine learning algorithm 3512 may generate the behavior modifying notification 3518 utilizing the behavior adherence data, demographic information 3520, health and behavior research data 3504, biometric data 3522 from a smart health device 3516, and location data 3536 from the user's mobile device. The smart health device 3516 may additionally provide physical activity data 3506 utilized by the behavior analyzer 3502. The planned activities data 3540 may be collected from the user's day planner or social media activity made available to the system 3500. The health and behavior research data 3504 is provided to the machine learning algorithm 3512 to enable the machine learning algorithm 3512 to identify target behaviors that may be changed to improve the health of the user 3524. The demographic information 3520 may be utilized to determine similar users and predict the success of a possible suggestion and modification to change the behavior of the user.

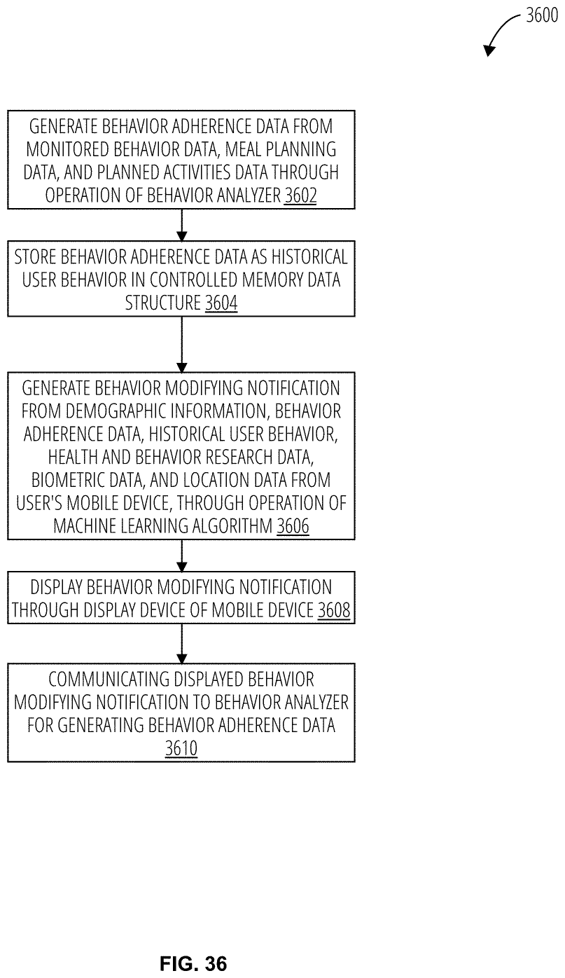

The system 3500 may be operated in accordance with the process described in FIG. 36.

Referencing FIG. 36, a method 3600 generates behavior adherence data from monitored behavior data (including user food log data), meal planning data, and planned activities data through operation of a behavior analyzer. In block 3604, method 3600 stores behavior adherence data as historical user behavior in a controlled memory data structure. In block 3606, method 3600 generates a behavior modifying notification from demographic information, the behavior adherence data, the historical user behavior, health and behavior research data, biometric data, and location data from a user's mobile device, through operation of a machine learning algorithm. In block 3608, method 3600 displays the behavior modifying notification (via various potential methods, including a standard pop-up, SMS, audio alarm, etc.) through a display device of the mobile device. In block 3610, method 3600 communicates displayed suggestions and modifications to the behavior analyzer for generating the behavior adherence data.

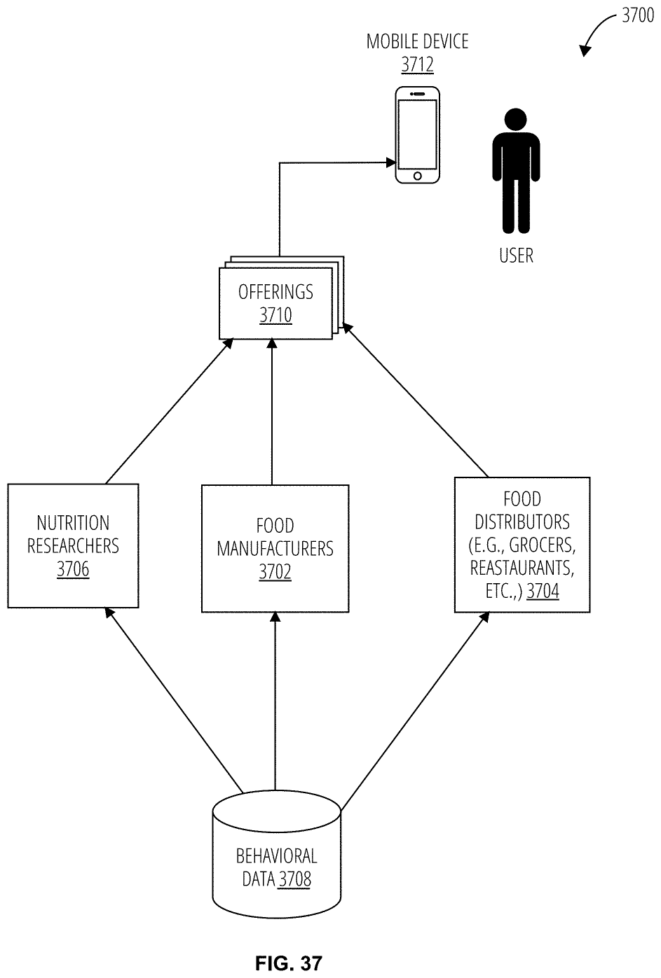

Referencing FIG. 37, a system 3700 is shown in accordance with one embodiment. The system 3700 illustrates behavioral data 3708 being utilized by nutrition researchers 3706, food manufacturers 3702, and food distributors 3704 to generate targeted offerings 3710 that may be communicated to a user's mobile device 3712. In some configurations, the food distributor 304 may be defined as any entity that is a source of food to grocers, other retailers, or directly to individuals in certain circumstances.

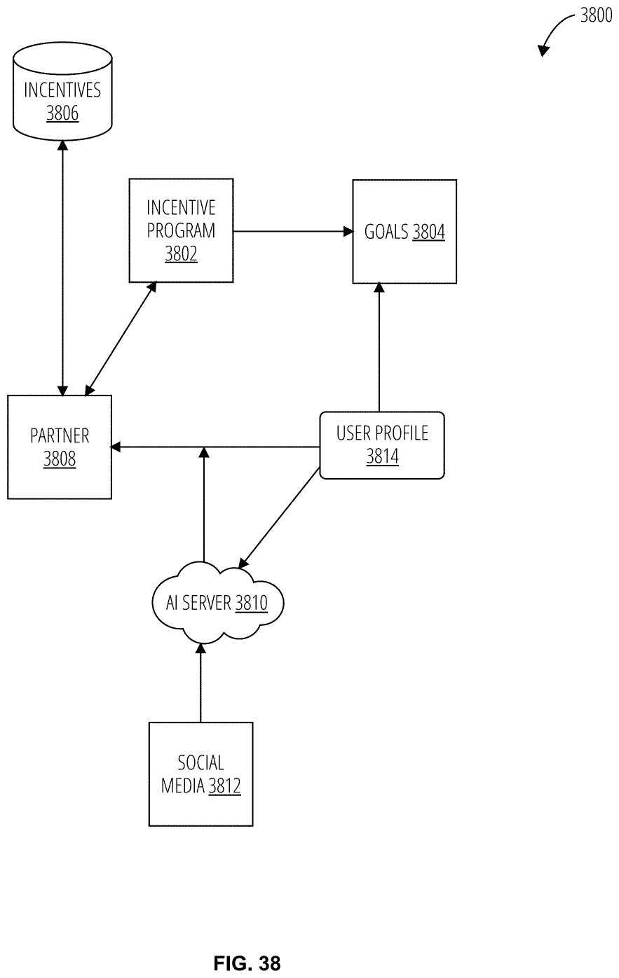

Referencing FIG. 38, a system 3800 is shown in accordance with one embodiment. The system 3800 illustrates a process of allowing a machine learning algorithm 3810 to identify and communicate information associated with a specific user profile 3814 to a plurality of advertising partners 3808 based on the user's social media activity 3812. The advertising partners 3808 may be provided with associated information from the user profile 3814 based on their currently running incentives 3806. The incentives 3806 may allow the advertising partners 3808 to offer an incentive program 3802 to a user profile 3814 based on goals 3804 and social media activity 3812. In some configurations, the user profile 3814 may include food preferences (i.e., likes/dislikes), food restrictions (e.g., gluten free), health objectives (e.g., lose weight), budget, preferred brands and/or private labels, and preferred grocers and/or food distributors that may be factored into the machine learning algorithm to generate a behavior modification notification.

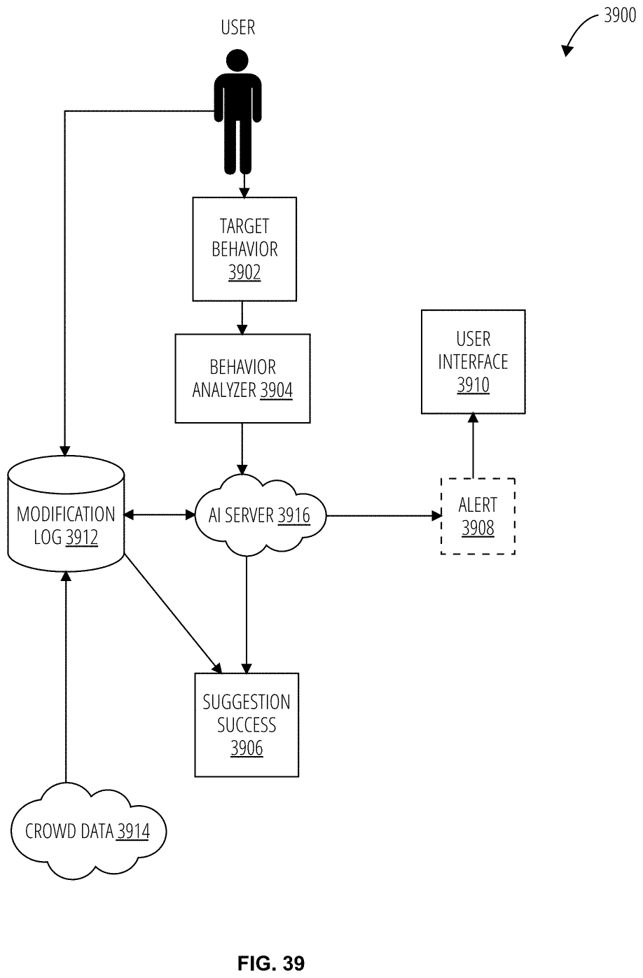

Referencing FIG. 39, a system 3900 is shown in accordance with one embodiment. A behavior analyzer 3904 of the system 3900 may detect a target behavior 3902 from the user. The behavior analyzer 3904 communicates the detection of the target behavior 3902 to the machine learning algorithm 3916. The machine learning algorithm 3916 may generate a behavior modifying notification 3908 based in part on the suggestion success 3906 of the behavior modifying notification 3908. The suggestion success 3906 may be determined by the machine learning algorithm 3916 by referencing a modification log 3912 comprising historical user behavior from the current user and similar users' behavior data 3914. The behavior modifying notification 3908 is then displayed through a user interface 3910. In some configurations, the desired behavior change caused by the behavior modifying notification 3908 may be stored in the modification log 3912 to determine the suggestion success 3906 of future alerts.

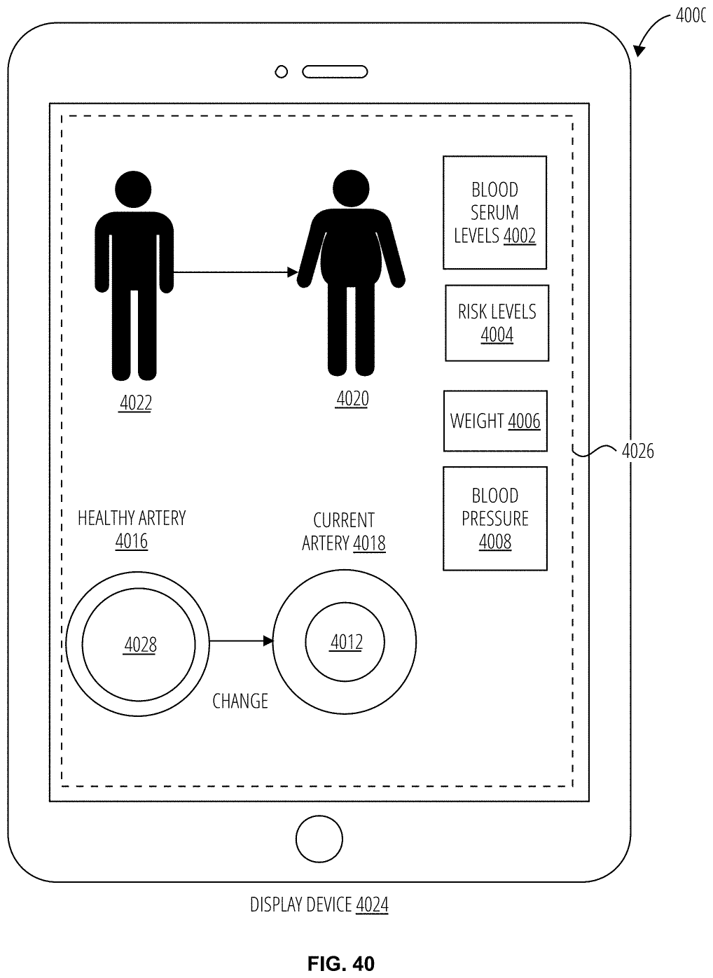

FIG. 40 illustrates a system 4000 in accordance with one embodiment. The system 4000 illustrates a display device 4024 showing a behavior modifying notification 4026 to suggest and modify a user's behavior. The behavior modifying notification 4026 shows a representative healthy user avatar 4022 compared to a current representation of the user's avatar 4020. The behavior modifying notification 4026 may show the current user's blood serum levels 4002, risk levels 4004, current weight 4006, and blood pressure 4008. The behavior modifying notification 4026 may also show a comparison of a healthy artery 4016 compared to the user's current artery 4018. The comparison may also show how the change in diet affected the user by showing the simulated change between a healthy diameter 4028 to the current diameter 4012 of the user's arteries.

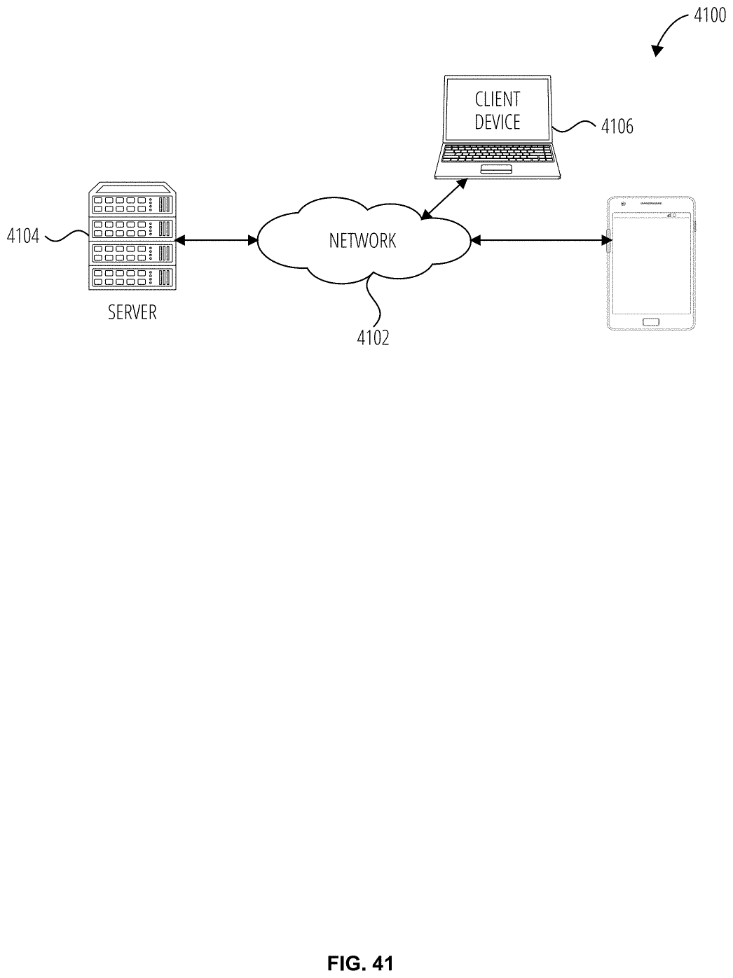

FIG. 41 illustrates a system 4100 in which a server 4104 and a client device 4106 are connected to a network 4102.

In various embodiments, the network 4102 may include the Internet, a local area network (“LAN”), a wide area network (“WAN”), and/or other data network. In addition to traditional data-networking protocols, in some embodiments, data may be communicated according to protocols and/or standards including near field communication (“NFC”), Bluetooth, power-line communication (“PLC”), and the like. In some embodiments, the network 4102 may also include a voice network that conveys not only voice communications, but also non-voice data such as Short Message Service (“SMS”) messages, as well as data communicated via various cellular data communication protocols, and the like.

In various embodiments, the client device 4106 may include desktop PCs, mobile phones, laptops, tablets, wearable computers, or other computing devices that are capable of connecting to the network 4102 and communicating with the server 4104, such as described herein.

In various embodiments, additional infrastructure (e.g., short message service centers, cell sites, routers, gateways, firewalls, and the like), as well as additional devices may be present. Further, in some embodiments, the functions described as being provided by some or all of the server 4104 and the client device 4106 may be implemented via various combinations of physical and/or logical devices. However, it is not necessary to show such infrastructure and implementation details in FIG. 41 to describe an illustrative embodiment.

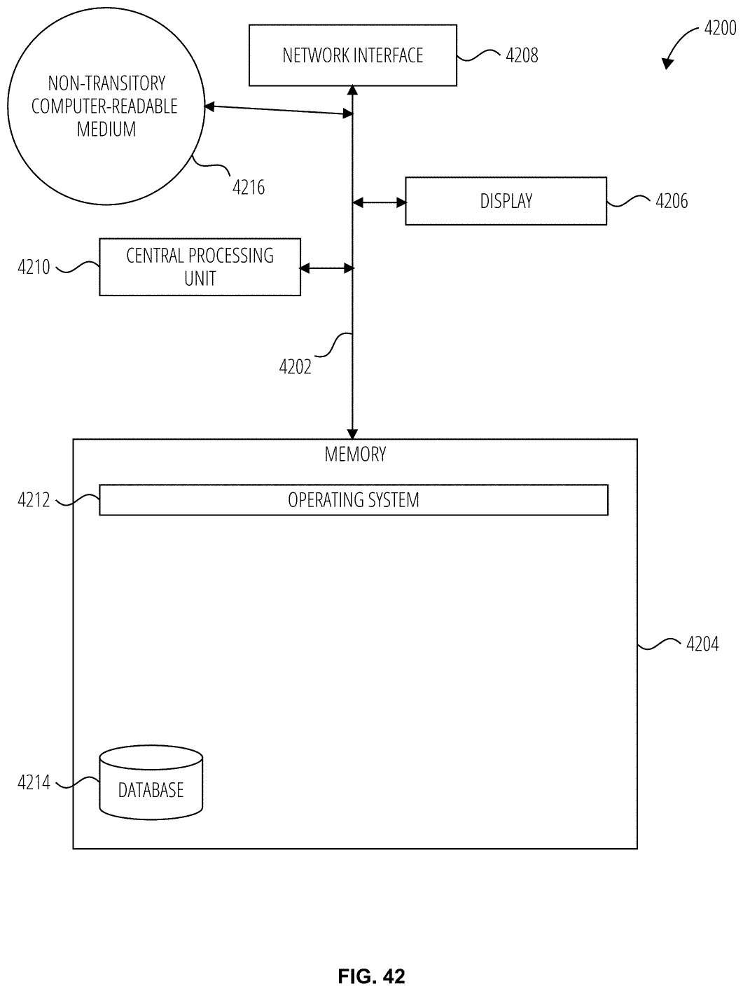

FIG. 42 illustrates several components of an exemplary system 4200 in accordance with one embodiment. In various embodiments, system 4200 may include a desktop PC, server, workstation, mobile phone, laptop, tablet, set-top box, appliance, or other computing device that is capable of performing operations such as those described herein. In some embodiments, system 4200 may include many more components than those shown in FIG. 42. However, it is not necessary that all of these generally conventional components be shown in order to disclose an illustrative embodiment. Collectively, the various tangible components or a subset of the tangible components may be referred to herein as “logic” configured or adapted in a particular way, for example as logic configured or adapted with particular software or firmware.

In various embodiments, system 4200 may comprise one or more physical and/or logical devices that collectively provide the functionalities described herein. In some embodiments, system 4200 may comprise one or more replicated and/or distributed physical or logical devices.

In some embodiments, system 4200 may comprise one or more computing resources provisioned from a “cloud computing” provider, for example, Amazon Elastic Compute Cloud (“Amazon EC2”), provided by Amazon.com, Inc. of Seattle, Wash.; Sun Cloud Compute Utility, provided by Sun Microsystems, Inc. of Santa Clara, Calif.; Windows Azure, provided by Microsoft Corporation of Redmond, Wash., and the like.

System 4200 includes a bus 4202 interconnecting several components including a network interface 4208, a display 4206, a central processing unit 4210, and a memory 4204.

Memory 4204 generally comprises a random access memory (“RAM”) and permanent non-transitory mass storage device, such as a hard disk drive or solid-state drive. Memory 4204 stores an operating system 4212.

These and other software components may be loaded into memory 4204 of system 4200 using a drive mechanism (not shown) associated with a non-transitory computer-readable medium 4216, such as a DVD/CD-ROM drive, memory card, network download, or the like.

Memory 4204 also includes database 4214. In some embodiments, system 4200 may communicate with database 4214 via network interface 4208, a storage area network (“SAN”), a high-speed serial bus, and/or via the other suitable communication technology.

In some embodiments, database 4214 may comprise one or more storage resources provisioned from a “cloud storage” provider, for example, Amazon Simple Storage Service (“Amazon S3”), provided by Amazon.com, Inc. of Seattle, Wash., Google Cloud Storage, provided by Google, Inc. of Mountain View, Calif., and the like.

Terms used herein should be accorded their ordinary meaning in the relevant arts, or the meaning indicated by their use in context, but if an express definition is provided, that meaning controls.

“Circuitry” refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” refers to software logic embodied as processor-executable instructions stored in read-only memories or media.

“Hardware” refers to logic embodied as analog or digital circuitry.

“Logic” refers to machine memory circuits, non transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

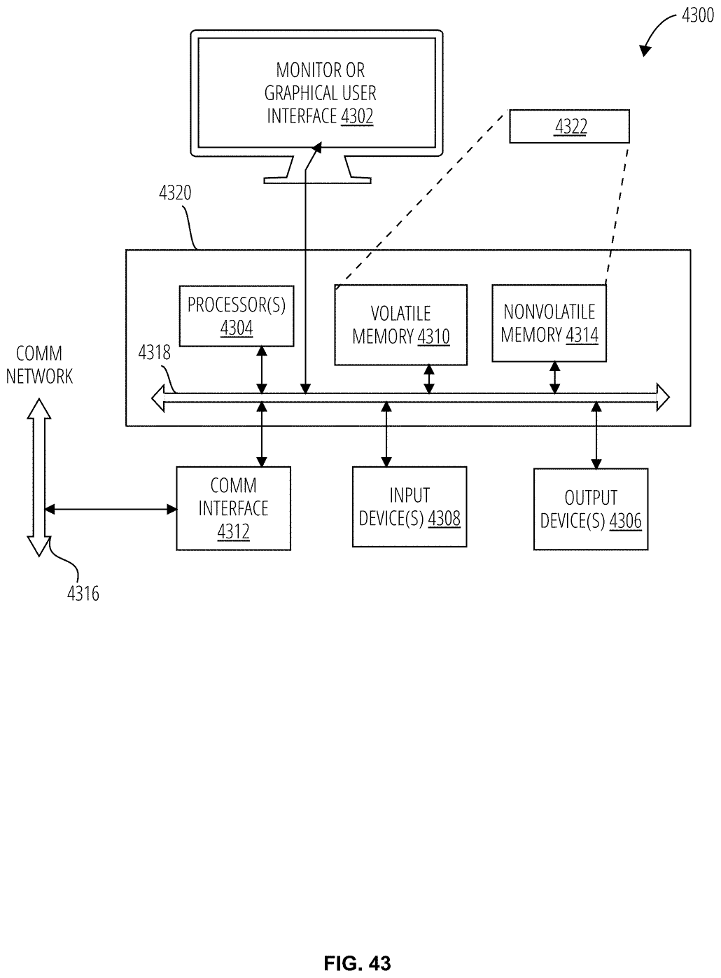

FIG. 43 is an example block diagram of a computing device 4300 that may incorporate embodiments of the present invention. FIG. 43 is merely illustrative of a machine system to carry out aspects of the technical processes described herein, and does not limit the scope of the claims. One of ordinary skill in the art would recognize other variations, modifications, and alternatives. In one embodiment, the computing device 4300 typically includes a monitor or graphical user interface 4302, a data processing system 4320, a communication network interface 4312, input device(s) 4308, output device(s) 4306, and the like.

As depicted in FIG. 43, the data processing system 4320 may include one or more processor(s) 4304 that communicate with a number of peripheral devices via a bus subsystem 4318. These peripheral devices may include input device(s) 4308, output device(s) 4306, communication network interface 4312, and a storage subsystem, such as a volatile memory 4310 and a nonvolatile memory 4314.

The volatile memory 4310 and/or the nonvolatile memory 4314 may store computer-executable instructions and thus forming logic 4322 that when applied to and executed by the processor(s) 4304 implement embodiments of the processes disclosed herein.

The input device(s) 4308 include devices and mechanisms for inputting information to the data processing system 4320. These may include a keyboard, a keypad, a touch screen incorporated into the monitor or graphical user interface 4302, audio input devices such as voice recognition systems, microphones, and other types of input devices. In various embodiments, the input device(s) 4308 may be embodied as a computer mouse, a trackball, a track pad, a joystick, wireless remote, drawing tablet, voice command system, eye tracking system, and the like. The input device(s) 4308 typically allow a user to select objects, icons, control areas, text and the like that appear on the monitor or graphical user interface 4302 via a command such as a click of a button or the like.

The output device(s) 4306 include devices and mechanisms for outputting information from the data processing system 4320. These may include the monitor or graphical user interface 4302, speakers, printers, infrared LEDs, and so on as well understood in the art.

The communication network interface 4312 provides an interface to communication networks (e.g., communication network 4316) and devices external to the data processing system 4320. The communication network interface 4312 may serve as an interface for receiving data from and transmitting data to other systems. Embodiments of the communication network interface 4312 may include an Ethernet interface, a modem (telephone, satellite, cable, ISDN), (asynchronous) digital subscriber line (DSL), FireWire, USB, a wireless communication interface such as Bluetooth or WiFi, a near field communication wireless interface, a cellular interface, and the like.

The communication network interface 4312 may be coupled to the communication network 4316 via an antenna, a cable, or the like. In some embodiments, the communication network interface 4312 may be physically integrated on a circuit board of the data processing system 4320, or in some cases may be implemented in software or firmware, such as “soft modems”, or the like.

The computing device 4300 may include logic that enables communications over a network using protocols such as HTTP, TCP/IP, RTP/RTSP, IPX, UDP and the like.

The volatile memory 4310 and the nonvolatile memory 4314 are examples of tangible media configured to store computer readable data and instructions to implement various embodiments of the processes described herein. Other types of tangible media include removable memory (e.g., pluggable USB memory devices, user device SIM cards), optical storage media such as CD-ROMS, DVDs, semiconductor memories such as flash memories, non-transitory read-only-memories (ROMS), battery-backed volatile memories, networked storage devices, and the like. The volatile memory 4310 and the nonvolatile memory 4314 may be configured to store the basic programming and data constructs that provide the functionality of the disclosed processes and other embodiments thereof that fall within the scope of the present invention.

Logic 4322 that implements embodiments of the present invention may be stored in the volatile memory 4310 and/or the nonvolatile memory 4314. Said logic 4322 may be read from the volatile memory 4310 and/or nonvolatile memory 4314 and executed by the processor(s) 4304. The volatile memory 4310 and the nonvolatile memory 4314 may also provide a repository for storing data used by the logic 4322.

The volatile memory 4310 and the nonvolatile memory 4314 may include a number of memories including a main random access memory (RAM) for storage of instructions and data during program execution and a read only memory (ROM) in which read-only non-transitory instructions are stored. The volatile memory 4310 and the nonvolatile memory 4314 may include a file storage subsystem providing persistent (non-volatile) storage for program and data files. The volatile memory 4310 and the nonvolatile memory 4314 may include removable storage systems, such as removable flash memory.

The bus subsystem 4318 provides a mechanism for enabling the various components and subsystems of data processing system 4320 communicate with each other as intended. Although the communication network interface 4312 is depicted schematically as a single bus, some embodiments of the bus subsystem 4318 may utilize multiple distinct busses.

It will be readily apparent to one of ordinary skill in the art that the computing device 4300 may be a device such as a smartphone, a desktop computer, a laptop computer, a rack-mounted computer system, a computer server, or a tablet computer device. As commonly known in the art, the computing device 4300 may be implemented as a collection of multiple networked computing devices. Further, the computing device 4300 will typically include operating system logic (not illustrated) the types and nature of which are well known in the art.

Terms used herein should be accorded their ordinary meaning in the relevant arts, or the meaning indicated by their use in context, but if an express definition is provided, that meaning controls.

“Circuitry” refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” refers to software logic embodied as processor-executable instructions stored in read-only memories or media.

“Hardware” refers to logic embodied as analog or digital circuitry.

“Logic” refers to machine memory circuits, non transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

What is claimed is:

1. A method of operating a machine learning model on a menu generation platform, comprising:
storing user modification for a menu generation platform comprising a menu generation algorithm, a food preference algorithm, a geofenced equivalence recommendations algorithm, and a behavior modification algorithm in a historical user interaction database;
generating modification and optimization controls for the menu generation platform through operation of a machine learning algorithm configured by entries in the historical user interaction database and entries in a platform user interaction database; and
generating relevant suggestions and modifications for the menu generation algorithm, the food preferences algorithm, geofence equivalence algorithm, and behavior modification algorithm, displayable through a user interface.

2. The method of operating a machine learning model on a menu generation platform as in claim 1, further comprising storing user data including current health, daily kcal targets, location information, and dietary restrictions.

3. The method of operating a machine learning model on a menu generation platform as in claim 2, further comprising receiving medical conditions and medication requirements of a user and wherein generating relevant suggestions and modifications are based at least in part on the medical conditions and medication requirements of the user.

4. The method of operating a machine learning model on a menu generation platform as in claim 1, further comprising generating a shopping list based at least in part on a food menu generated by the menu generation algorithm.

5. A method, comprising:
retrieving at least one user profile, the at least one user profile comprising user preferences;
inputting, into a menu generation algorithm, the user preferences;
retrieving, based at least in part on the user preferences, food items from at least one food database;
generating, based at least in part on the food items, a meal plan comprising one or more meals;
determining a portion size associated with the one or more meals; and
generating a food menu.

6. The method of claim 5, wherein the user profile comprises one or more of health information, health goals, food preferences, demographic information, location information, and preferred grocer information.

7. The method of claim 6, wherein the preferred grocer information comprises an identified grocer and the method further comprises generating a shopping list associated with the food menu and the identified grocer.

8. The method of claim 5, further comprising receiving input from a user associated with the user profile, the input associated with modifying the food items.

9. The method of claim 8, wherein the input from the user comprises one or more of food likes, food dislikes, nutrient targets, dietary restrictions, financial budget, or kcal targets.

10. The method of claim 5, further comprising determining, based at least in part on the food menu, nutrition information associated with the one or more meals.

11. The method of claim 10, further comprising determining, based at least in part on the nutrition information associated with the one or more meals, nutrition deficiencies.

12. The method of claim 11, further comprising, based at least in part on the nutrition deficiencies, additional meals to address the nutrition deficiencies.

13. The method of claim 5, wherein retrieving the at least one or more user profile comprises retrieving two or more user profiles and generating the food menu comprises generating a menu for two or more users.

14. The method of claim 5, further comprising determining an activity level of a user and wherein generating a food menu is based, at least in part, on the activity level of the user.

15. The method of claim 5, further comprising:
generating a food log that contains data associated with the food menu; and
receiving input from a user indicating that the user has been consuming food according to the food menu.

16. The method of claim 15, wherein receiving input from a user further indicates consumed foods that are not associated with the food menu.

17. The method of claim 5, further comprising:
determining a grocery store in proximity to a user;
receiving availability information associated with food items available at the grocery store;
receiving pricing information associated with the food items available at the grocery store; and
wherein generating the food menu is based at least in part on the availability information and the pricing information.

18. The method of claim 5, further comprising receiving a list of menu items from a restaurant and wherein generating the food menu comprises recommending one or more meals from the restaurant.

19. A method of generating a menu, comprising:
receiving a user profile associated with a user comprising one or more of a location, a budget, a food dislike, a food preference, a health target, a health condition, a nutritional target, a kcal target, and an activity level associated with the user;
generating, based on the user profile, a food menu;
receiving, through a user interface of a computing device associated with the user, a modification to the food menu;
determining, based on the modification to the food menu, a change to nutritional information or caloric information; and
generating, based on the change to nutritional information or caloric information, a second food menu comprising the modification to the food menu, the second food menu generated to meet one or more of a nutritional target or a kcal target.

20. The method of generating a menu as in claim 19, further comprising receiving historical food menu data from a second user and generating the food menu is based, at least in part, on the historical food menu data from the second user.
